(19) United States
(12) Patent Application Publication
(10) Pub. No.: US 2015/0132345 A1
(43) Pub. Date: May 14, 2015

(54) NITRIC OXIDE-SEQUESTERING TOPICAL FORMULATIONS

(71) Applicants: Yossef Av-Gay, Vancouver (CA); Horacio Bach, Vancouver (CA)

(72) Inventors: Yossef Av-Gay, Vancouver (CA); Horacio Bach, Vancouver (CA)

(21) Appl. No.: 14/117,045

(22) PCT Filed: May 9, 2012
(86) PCT No.: PCT/IL12/50161

§ 371 Date: Nov. 12, 2013

Related U.S. Application Data
(60) Provisional application No. 61/483,762, filed on May 9, 2011.

(51) Int. Cl. A61K 33/00; A61K 9/70; A61Q 17/00; A61K 9/06; A61K 8/02; A61Q 19/00; A61K 8/19; A61K 9/08

(52) U.S. Cl. CPC A61K 33/00; A61K 8/19; A61K 9/7007; A61K 9/08; A61K 9/06; A61K 8/0208; A61Q 19/00; A61Q 17/005; A61K 2800/87; A61K 2800/22
USPC 424/401; 424/443; 424/718; 222/630; 206/524.1

(57) ABSTRACT

Topical formulations, comprising a carrier having gaseous nitric oxide sequestered therein, and optionally further comprising a pharmaceutically, cosmetic or cosmeceutically active agent, which are useful is medical, cosmetic or cosmeceutic applications, are provided herein. Systems, devices and processes for obtaining such formulations are also disclosed.

### FIELD AND BACKGROUND OF THE INVENTION

The present invention, in some embodiments thereof, relates to medical, cosmetic or cosmeceutic products and, more particularly, but not exclusively, to nitric oxide-sequestering topical formulations, to processes and systems for producing such formulations and to uses thereof.

Nitric oxide (NO) is a small, naturally produced, hydrophobic, free-radical gas that has a major role in innate immunity. NO exhibits broad reactivity and rapid diffusive properties through biological liquids and lipid membranes, with a short half-life in a physiological milieu [Subczynski and Wisniewska. 2000. Acta Biochim Pol. 47:613-625]. Overproduction of NO induced by the enzymatic activity of inducible nitric oxide synthase (iNOS) in various cell types has been shown to play a vital role in several inflammatory and immunoregulatory processes. NO has been shown to play important roles in vasodilatation, neurotransmission, angiogenesis, modulation of wound healing, and nonspecific responses to infection.

Nitric oxide is also known to function as an antimicrobial agent. Gaseous nitric oxide at a concentration of about 200 ppm has been demonstrated to clear pneumonia caused by pathogens such as Pseudomonas aeruginosa or S. aureus [McMullin et al., 2005, Respir. Care 5:1451-1456]. Topical applications of gaseous nitric oxide at about 200 ppm has been demonstrated to inhibit or prevent growth of a variety of microbial pathogens including P. aeruginosa, S. aureus, E. coli, Streptococcus spp. and Candida albicans [Ghaffari et al., 2005, Nitric Oxide, 14:21-29]. Nitric oxide has also been demonstrated to inhibit replication of a variety of viral pathogens including influenza virus, retroviruses, rhabdoviruses e.g. vesicular stomatitis virus, flavivirus e.g. Japanese Encephalitis virus [Rimmelzwaan et al., 1999, J. Virol 73(10): 8880-8883 and references cited therein].

NO has been shown to be bacteriostatic and bactericidal. Miller et al. [Nitric Oxide 20: 16-23, 2009] demonstrated that multiple 30 minutes treatments of 160 part per million (ppm) nitric oxide resulted in over a 5 log 10 colony forming unit per milliliter (CFU/ml) decrease in the bacterial load of Staphylococcus aureus, Escherichia coli and Pseudomonas aeruginosa.

A variety of methodologies have been attempted to deliver and study NO therapeutically. These include the use of polymers containing a diazeniumdiolate NO donor [Hetrick and Schoenfisch. 2007. Biomaterials 28:1948-1956; Nablo et al. 2005. Biomaterials 26:6984-6990], exposure chambers for direct topical application of NO [Ghaffari et al. 2006. Nitric Oxide 14:21-29; McMullin et al. 2005. Respir. Care 50:1451-1456], and filling a urinary catheter retention balloon with nitrite and ascorbic acid to release NO [Carlsson et al. 2005. Antimicrob. Agents Chemother. 49:2352-2355].

Nitric oxide-producing agents have been used to keep various medical and other devices substantially free of bacteria by their capacity to release nitric oxide under certain conditions. For example, U.S. Pat. Nos. 5,525,357, 7,105,502, 7,122,529 and 7,829,553, U.S. Patent Application Nos. 2003/1039697, 2004/0247640, 2006/0008529 and 20070196327, and International Patent Application Nos. WO 1995/024908, WO 2006/037105, WO 2007/1028657, WO 2002/1056864, WO 2006/1084910 and WO 2006/1078786, teach various industrial materials and polymers comprising nitric oxide precursors, which are capable of generating some amount of NO in situ when the material comes in contact with physiological media, or upon other chemical or physical stimuli.

Many NO-donors are thermally unstable, resulting in uncontrolled release of NO, unpredictable concentration, and unpredictable duration of effect. Nanoparticle formulations containing NO-donors stored in a dry matrix and released upon exposure to moisture have been investigated in the treatment of skin infections. However, the efficacy of these NO-releasing compositions depends on the type and amount of the ingredients that release or protect the NO-donors as well as thermal and biological conditions. Moreover, some of these topical formulations are toxic or inflammatory to the skin, while others generate NO₂, a noxious gas with negative effects on the respiratory.

Currently available topical NO-releasing formulations have demonstrated promising therapeutic capabilities in numerous medicinal and pharmaceutical applications. For example, NO-releasing topical ointments were effective in killing or inhibiting a wide range of bacteria, viruses, fungi and yeasts associated with skin infections. Gel formulations containing NO-donors have been demonstrated to possess antibacterial activity against E. coli, C-P. aeruginosa, B. subitilis and S. aureus as well as significant anti-inflammatory activity. Acidified nitrite cream treated tinea pedis (athlete's foot) effectively, while other topical NO-releasing creams treated molluscum contagiosum effectively. Topical NO has shown excellent therapeutic potential in sexual dysfunction by promoted arterial dilatation of the clitoris and in the treatment of impotence due to NO's action on smooth muscle relaxation of the corpus cavernosum. NO-releasing has also been found to exert estrogen-like beneficial effects on bone without the adverse systemic effects of estrogen. NO-donor containing ointments have been shown to prevent or reverse estrogen-depleted osteoporosis effectively in rodent models and human subjects.

At very low concentrations (up to 0.1 parts per million in air), gaseous nitric oxide (gNO) may be administered to humans having breathing problems since it was found to have beneficial effects due to its bronchodilatory and vasodilatory activity. However, still being somewhat toxic to humans, nitric oxide is rather complicated for use as a gas. Moreover, colorless gaseous NO (under some conditions) may react rapidly with atmospheric oxygen, yielding nitrogen dioxide (NO₂), a reddish-brownish gas with much higher toxicity than NO.

Gaseous nitric oxide (gNO), has been considered as a stable, highly antimicrobial delivery modality with little toxicity to human skin cells, for providing NO to target areas in desired amounts, at desired release rates and durations, without NO₂ generation or systemic effects [Ghaffari A, J. R., Wound Repair Regen., 2007, 15 (3), 368-77; and Ghaffari A, M. C., Nitric Oxide, 2006, 14 (1), 21-9]. However, the use of gNO for topical applications has been limited by its delivery and handling requirements [Englander L, F. A., J Clin Aesthet Dermatol., 2010, 3 (6), 45-50]. Additional background art includes U.S. Pat. No. 7,820,420 and U.S. Patent Application Nos. 20020168424, 20020182162, 20040014188, 20080069848, 20080069863, 20080311163, 20090148482, 20100016790, 20100098733, 20100297200 and 20110033437, as well as references cited hereinbelow.

### SUMMARY OF THE INVENTION

According to an aspect of some embodiments of the present invention there is provided a topical formulation comprising a carrier having gaseous nitric oxide sequestered therein.

According to some embodiments of the invention, an amount of sequestered gaseous nitric oxide is at least 0.01 μM NO₂ per gram of the formulation.

According to some embodiments of the invention, an amount of sequestered gaseous nitric oxide ranged from 0.01 to 5 μM per gram of the formulation.

According to some embodiments of the invention, the carrier is a pharmaceutically, cosmeceutic or cosmetic acceptable carrier.

According to some embodiments of the invention, the formulation further comprises a pharmaceutically, cosmetically or cosmeceutically active agent.

According to some embodiments of the invention, the active agent is an antimicrobial agent.

According to some embodiments of the invention, the carrier comprises at least one additive or excipient.

According to some embodiments of the invention, the at least one additive or excipient is selected from the group consisting of a mineral oil, a petrolatum, a tissue-specific (specialty) carrier, a surfactant system (stabilizer), a solubilizer, a tonicity enhancing agent, a structure-forming agent, a suspending agent, a gel-forming agent, a thickening agent, a pH adjusting agent (a buffer substance), a preservative, a penetration enhancer, a complexing agent, a lubricant, a demulcent, a viscosity enhancer and any combination of the forgoing.

According to some embodiments of the invention, the additive or excipient is hydrophobic.

According to some embodiments of the invention, the formulation is in a form of a cream, a lotion, an ointment, a wipe, a sponge, a solution, an emulsion, a paste, a gel, a patch, a pladget, a swab, a dressing and a pad.

According to some embodiments of the invention, the formulation is identified for use in the treatment of a medical condition treatable by topical or transdermal administration.

According to as aspect of some embodiments of the present invention there is provided a use of the formulation described herein, in the treatment of a medical condition treatable by topical or transdermal administration.

According to as aspect of some embodiments of the present invention there is provided a method of treating a medical condition treatable by topical or transdermal administration, the method comprising topically applying the formulation as described herein to a skin or mucosal tissue of a subject afflicted by the medical condition.

According to some embodiments of the invention, the medical condition is a microbial infection.

According to an aspect of some embodiments of the present invention there is provided a process of preparing the formulation as described herein, the process comprising exposing a topical formulation to a gaseous nitric oxide-containing environment.

According to some embodiments of the invention, the exposing comprises:

placing a topical formulation in a chamber; and

filling the chamber with the nitric oxide-containing environment.

According to some embodiments of the invention, the process further comprises, prior to the filling, generating a reduced pressure in the chamber.

According to some embodiments of the invention, the topical formulation comprises a carrier capable of sequestering therein at least 0.01 μM gaseous nitric oxide per gram.

According to some embodiments of the invention, the carrier comprises at least one additive or excipient.

According to some embodiments of the invention, the at least one additive or excipient is selected from the group consisting of a mineral oil, a petrolatum, a surfactant, a stabilizing agent, a structure-forming agent, a gel-forming agent, a gel-stabilizing agent, a thickening agent, and a suspending agent.

According to some embodiments of the invention, the additive or excipient is hydrophobic.

According to some embodiments of the invention, the topical formulation is in a form selected from the group consisting of a cream, a lotion, an ointment, a wipe, a sponge, a solution, an emulsion, a paste, a gel, a patch, a pladget, a swab, a dressing and pad.

According to an aspect of some embodiments of the present invention there is provided a charging device comprising:

a chamber comprising an inlet for receiving a gaseous nitric-oxide containing environment and an outlet for releasing the gaseous nitric-oxide containing environment; and

a topical formulation disposed within the chamber.

According to some embodiments of the invention, the device further comprises a desiccant configured to absorb humidity from the gaseous environment.

According to some embodiments of the invention, the device further comprises a nitric oxide indicator configured to undergo a color change suitable for visual assessment of whether the formulation has been exposed to the nitric oxide.

According to some embodiments of the invention, the device further comprises an outlet for generating a reduced pressure in the chamber.

According to some embodiments of the invention, the device further comprises a package enclosing the topical formulation.

According to as aspect of some embodiments of the invention there is provided a charging device comprising:

a sealed chamber having a reduced pressure therewithin; and

a topical formulation disposed within the chamber.

According to some embodiments of the invention, the chamber further comprises an outlet for generating the negative pressure within the chamber.

According to some embodiments of the invention, the device further comprises an inlet configured for receiving a gaseous nitric oxide-containing environment and an outlet for releasing the gaseous nitric oxide-containing environment.

According to some embodiments of the invention, the device further comprises a package enclosing the topical formulation.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a schematic illustration of an exemplary charging device 500, according to some embodiments of the present invention, having a chamber 137, inlet 125 with valve 11, inlet 135 with valve 2, and inlet 145 with valve 15, inlets which may share a common tube 23 with valve 19 that connects to chamber 137; outlet 126 with valve 36 and connect to purge flow rotomoter 46, outlet 136 with valve 41 and connect to purge flow rotomoter 45, outlets which may share a common tube 33 that connects to chamber 137;

FIG. 2 presents comparative plots demonstrating nitric oxide release from commercial topical formulations upon exposing the products to 2% nitric oxide for 24 hours in a sealed stainless steel chamber and measuring release of nitric oxide at 24, 48 and 72 hours using Griess reagent (results are expressed per gram of product, showing the mean±SD of two independent experiments);

FIGS. 3A-F present the results obtained from NO-release experiments conducted for commercial cream products which were exposed to 2% nitric oxide for 24 hours in a sealed stainless steel chamber, according to some embodiments of the present invention, wherein nitric oxide release was measured one year after exposure, at time intervals of 4, 8 and 12 hours, wherein FIG. 3A presents the NO release profile obtain for NO-impregnated Benadryl™, FIG. 3B for Canesten™, FIG. 3C for Hydrosone™, FIG. 3D for Gold Bond™, FIG. 3E for Lanacane™, FIG. 3F for Vagisil™ and the results are expressed in μM of nitrite per gram of product (shown are mean values±standard deviation of three independent experiments); and

FIG. 4 presents images demonstrating the antibacterial activity of Hydrosone treated with 2% nitric oxide for 24 hours, aseptically deposited at the bottom of a 96-well plate by centrifugation at 800 rpm for 3 minutes, and contacted with serial dilutions of Pseudomonas aeruginosa culture, wherein the appearance of a greenish color in the culture of the microorganism exposed to non-treated Hydrosone indicates a normal growth cause by the release of iron-binding molecules secreted by P. aeruginosa to the medium, and showing that growth was not observed in cultures exposed to NO-impregnated Hydrosone.

### DESCRIPTION OF SPECIFIC EMBODIMENTS OF THE INVENTION

The present invention, in some embodiments thereof, relates to medical, cosmetic or cosmeceutic products and, more particularly, but not exclusively, to nitric oxide-sequestering topical formulations, to processes and systems for producing such formulations and to uses thereof.

The present inventors have contemplated charging nitric oxide into topical formulations, with the aim of imparting anti-bacterial activity to the formulations. As demonstrated in the Examples section that follows, the present inventors have shown that nitric oxide was successfully charged into and released from a variety of commercially available creams, and that the gNO-treated formulations remained stable and capable of releasing gNO also after one year. The present inventors have also shown that the gNO-treated formulations exhibited antibacterial activity.

Hence, according to an aspect of embodiments of the present invention, there is provided a topical formulation which includes a carrier having gaseous nitric oxide (gNO) sequestered therein. The formulation presented herein is generally referred to herein and throughout as a “gNO-sequestering topical formulation”.

For clarity, it is noted that the term “sequestering” in the context of gaseous nitric oxide (gNO), and the terms “impregnated”, “embedded”, “charged”, “loaded”, “dosed” and “treated” are used interchangeably hereinbelow and throughout to denote a topical formulation releasably sequestering gNO therein.

As used herein, the term “sequestering” and any grammatical inflection thereof refer to a state of a topical formulation having a foreign substance, such as a gas, incorporated therein; a state which exists substantially from the time the substance is introduced into the formulation from an external source to the time the substance leaves the formulation. According to some embodiments of the present invention, in the chemical sense, the sequestered substance being released from the formulation is essentially the same substance that was charged into the topical formulation.

According to some embodiments of the present invention, the topical formulation is releasably sequestering gNO, and the term “sequestering” therefore encompasses the phrase “releasably sequestering”.

Hence, “releasably sequestering”, as used herein, is meant to define a formulation having gNO absorbed therein in a reversible manner, wherein the gNO can be released to the ambient environment from the formulation under certain conditions. According to some embodiments of the present invention, gNO is released from the gNO-sequestering topical formulation at an essentially controllable manner.

As used herein, the phrase “topical formulation” describes a preparation which is intended for application onto a biologic a surface (a topical bodily site), e.g., a skin, scalp or mucosal tissue, and which is aimed at exhibiting an effect on the skin or mucosal tissue, or transdermally, by penetrating the skin or mucosa and entering into the system.

Topical formulations are typically used for topical or transdermal application of active agents, for exhibiting a therapeutic, cosmetic or cosmeceutic effect.

Hence, a topical formulation as described herein comprises a carrier, which can be a pharmaceutically, cosmetic or cosmeceutically acceptable carrier, as defined herein.

As used herein, the term “pharmaceutically, cosmetically or cosmeceutically acceptable carrier” describes a carrier or a diluent that does not cause significant irritation to an organism and does not abrogate the biological and/or chemical activity and/or properties of the applied ingredient(s).

In some embodiments, the carrier is such that provides the formulation with a consistency that is suitable for topical application.

Examples of acceptable carriers that are usable in the context of the present invention include carrier materials that are well-known for use in the cosmetic and medical arts as bases for e.g., emulsions, creams, aqueous solutions, oils, ointments, pastes, gels, lotions, milks, suspensions, swabs, pledgets, pads, dressings and the like, depending on the final form of the composition.

In some embodiments, the carrier used to concoct the NO-sequestering formulation described herein is selected and/or adjusted to make up a cream.

The topical formulation can be a commercially available carrier formulation or be a part of a commercially available pharmaceutical, cosmetic or cosmeceutic product which may further comprise an active ingredient. Alternatively, the topical formulation can include some of the components which are utilized to make up a pharmaceutical, cosmetic or cosmeceutic carrier or product, and can be later be incorporated into a final therapeutic, cosmetic or cosmeceutic product or admixed with additional components to make up the pharmaceutical, cosmetic or cosmeceutic carrier or product.

According to some embodiments of the present invention, the amount of gNO sequestered in the topical formulation is an amount sufficient to exhibit a therapeutic, cosmetic or cosmeceutic effect related to gaseous nitric oxide and hence can be regarded as a pharmaceutically, cosmeceutically or cosmetically effective amount.

The phrase “pharmaceutically, cosmetically or cosmeceutically effective amount” as used herein in the context of sequestered gNO describes an amount of a gNO that is sufficient to significantly induce a positive modification in the condition being treated, but low enough to avoid significant side effects, within the scope of sound judgment of the skilled artisan. The effective amount of the sequestered gNO may vary with the particular condition being treated, the particular skin or mucosal tissue onto which the formulation is applied, the age and physical condition of the biological subject being treated, the severity of the condition, the duration of the treatment, the nature of concurrent therapy, the particular pharmaceutically, cosmetically or cosmeceutically acceptable topical carrier utilized, and like factors within the knowledge and expertise of the skilled artisan.

The amount of sequestered gNO which can be impregnated into a topical formulation during a charging process, as described hereinbelow, depends, among other factors, on the volume of the processed sample, its surface area during the process, and the composition of the topical formulation.

As known in the art, different substances have different solubility in different media, and this general principle holds also in the case of the capacity of a substance to sequester gNO at any given conditions. Topical formulation can be selected to be made from ingredients having a high capacity to sequester gNO, or alternatively, one can select one or more ingredients generally suitable for topical formulations which is having a high capacity to sequester gNO, and which can be admixed into a topical formulation. Suitable ingredients for sequestering gNO in high capacity are further discussed hereinbelow.

According to some embodiments of the present invention, the amount of sequestered gNO is at least 0.01 μM gNO per one gram of the formulation.

According to some embodiments of the present invention, the amount of sequestered gNO is at least 0.02 μM gNO, at least 0.05 μM gNO, at least 0.08 μM gNO, at least 0.1 μM gNO, at least 0.15 μM gNO, at least 0.2 μM gNO, at least 0.25 μM gNO, at least 0.3 μM gNO, at least 0.35 μM gNO, at least 0.4 μM gNO, at least 0.45 μM gNO, at least 0.5 μM gNO, at least 0.6 μM gNO, at least 0.7 μM gNO, at least 0.8 μM gNO, at least 0.9 μM gNO, at least 1 μM gNO, at least 1.2 μM gNO, at least 1.5 μM gNO, at least 2 μM gNO, at least 2.5 μM, at least 3 μM gNO, at least 3.5 μM gNO, at least 4 μM gNO, at least 4.5 μM gNO, or at least 5 μM gNO, per one gram of the formulation, although higher amounts are also contemplated.

According to some embodiments of the present invention, the amount of sequestered gaseous nitric oxide ranges from 0.01 μM to 5 μM of gNO, or from 0.01 μM to 2.5 μM of gNO per one gram of the formulation, including any intermediate value or range therebetween.

In other embodiments, the amount of gNO sequestered in the topical formulation is an amount sufficient to exhibit a bacteriostatic effect, a stabilization effect, a sterilization effect and/or an anti-contamination effect. In such cases, gNO can be used as a preservative, and the effective amount can be regarded as an amount sufficient to prevent or reduce bacterial growth in the formulation. In these embodiments, the amount of sequestered gNO required for exhibiting a bacteriostatic effect, a stabilization effect and/or an anti-contamination effect can be as low as 10⁻⁴ μM, and hence can be, for example, at least 10⁻⁴ μM, at least 5×10⁻⁴ μM, at least 10⁻³ μM, at least 5×10⁻³, at least 0.01 μM, at least 0.05 μM, at least 0.1 μM, at least 0.5 μM, and up to 1 μM.

In some embodiments, the incorporation of sequestered gNO in a formulation provides a sterilization effect to the formulation. Such an effect is useful, for example, in cases where the carrier and/or active or non-active ingredients in the formulation are unstable under common sterilization conditions such as heat.

In general, the phrase “sufficient amount of sequestered gNO” refers to the above and encompasses, for example, a bacteriostatically effective amount, a stabilizing effective amount, a sterilizing effective amount, an anti-contamination effective amount, a pharmaceutically effective amount, a cosmetically effective amount and/or cosmeceutically effective amount. In some embodiments, such an amount can range from 10⁻⁴ μM to 5 μM, or is as indicated hereinabove.

According to some embodiments of the present invention, the gNO-sequestering formulation presented herein can be formulated by admixture of carriers, excipients and additives as described hereinbelow, which jointly serve as the main reservoir of the releasably sequestered gNO.

Thus, according to some embodiments of the present invention, at least some of the ingredients of the gNO-sequestering topical formulation presented herein are selected also to be suitable to sequester gNO. According to some embodiments of the present invention, most of the mass of the gNO-sequestering topical formulation is composed of ingredients which are suitable to sequester gNO, namely at least 50%, 60%, 70%, 80%, 90% or more of the mass of the formulation is composed of ingredients which are suitable to sequester gNO.

According to some embodiments, the gNO-sequestering topical formulation presented herein includes a pharmaceutically acceptable excipients and/or carriers, as those skilled in the art can select using conventional criteria. As used herein, the term “carrier” refers to a base which is the main ingredient of a mixture.

The carrier can be selected from the known carriers for topical formulations which include, but are not limited to, petroleum derivatives such as mineral oil and white petrolatum, silicon oil, polyethers such as polyethylene glycol, polyvinyls such as polyvinyl alcohol and povidone, cellulose derivatives such as methylcellulose and hydroxypropyl methylcellulose, animal fats such as lanolin, polymers of acrylic acid such as carboxypolymethylene gel, vegetable fats such as peanut oil and polysaccharides such as dextrans, and glycosaminoglycans such as sodium hyaluronate, saline solutions, water and combinations thereof.

In the context of embodiments of the present invention, gNO can be sequestered in hydrophobic carriers to a greater extent than in hydrophilic carries, although hydrophilic carries can be impregnated with gNO as well.

According to some embodiments, the carrier is hydrophobic or the formulation comprises one or more hydrophobic substance(s) in an amount sufficient for forming a hydrophobic carrier. According to some embodiments of the invention, the one or more hydrophobic substance(s) make up at least 20% by weight, or at least 30%, at least 40%, at least 50%, at least 60%, at least 70%, at least 80%, at least 90% and can also be 100% by weight of the total weight of the carrier.

Hydrophobic carriers or substances that can be used to make up a carrier include, without limitation, petroleum-based carriers, mineral oils such as silicon and/or silicon oil-containing carriers, liquid and semi-solid paraffins, lanolin, beeswax, vegetable oil, glycerin monostearate, ethyl oleate, higher alcohols, polyethylene glycol, mono- and poly-unsaturated oils and fats from plant or animal sources, hardened silicone oils, hardened mineral oils and combinations thereof.

Hydrophilic and semi-hydrophillic carriers include, without limitation, vegetable oils or mineral oils comprising from 0.5% to 5% by weight hydroxyethyl-cellulose, carboxymethylcellulose, polyvinyl-pyrrolidone and other non-toxic hydrophilic polymers, such as, for example, cellulose derivatives, methyl-cellulose, alkali metal salts of carboxymethyl-cellulose, hydroxymethyl-cellulose, hydroxyethyl-cellulose, methylhydroxypropyl-cellulose and hydroxypropyl-cellulose, acrylates or methacrylates, such as salts of polyacrylic acid or ethyl acrylate, polyacrylamides, natural products, such as gelatin, alginates, pectins, chitin, chitosan, tragacanth, karaya gum, xanthan gum, carrageenin, carrageenan, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, cross-linked polyacrylic acid, such as neutral Carbopol, water, mixtures of water and water-miscible solvents such as C₁₋₇-alkanols (low alcohols), and any mixtures thereon.

The most commonly used hydrophilic carriers for topical formulations other than water include methyl-cellulose, alkali metal salts of carboxymethyl-cellulose, hydroxymethyl-cellulose, hydroxyethyl-cellulose, methylhydroxypropyl-cellulose and hydroxypropyl-cellulose, neutral Carbopol and any mixtures thereof.

According to some embodiments, the gNO-sequestering formulation presented herein further includes customary pharmaceutically acceptable excipients and additives known to the person skilled in the art, which may include, without limitation, tissue-specific (specialty) carriers, surfactant systems (stabilizers) effective to stabilize emulsions (e.g., oil-in-water emulsions and water-in-oil emulsions), solubilizers, tonicity enhancing agents, structure-forming agents, suspending agents, gel-forming agents, thickening agents, buffer substances, preservatives, thickeners, penetration enhancers, complexing agents, lubricants, demulcents, viscosity enhancers and other excipients.

According to some embodiments, a solubilizer can be used in the gNO-sequestering formulation presented herein, to solubilize any hydrophobic active ingredient in the formulation, including gNO. In some embodiments, a solubilizer is any substance or combination of substances that increases the amount of gNO which can be impregnated the formulation.

Exemplary suitable solubilizers include, for example, tyloxapol, fatty acid glycerol poly-lower alkylene glycol esters, fatty acid poly-lower alkylene glycol esters, polyethylene glycols, glycerol ethers, methylene, ethylene, 1,3-propylene, 1,2-propylene, 1,5-pentylene, 2,5-hexylene or 1,7-heptylene and any mixtures thereof.

Alternatively, in some embodiments, the solubilizer is selected as a substance or combination of substances that increases the solubility of other active ingredients in the formulation carrier.

The amount added is typically sufficient to solubilize the gNO and/or any active ingredient into the goal concentration. For example, the concentration of the solubilizer is from 0.1 to 5000 times the concentration of the gNO and/or any other active ingredient.

As pH can also affect gNO impregnation, a pH adjusting agent (a buffer substance) may also form an ingredient in the gNO-sequestering formulation presented herein. Examples of buffer substances include, without limitation, acetate, ascorbate, borate, hydrogen carbonate/carbonate, citrate, gluconate, lactate, phosphate, propionate and TRIS (tris(hydroxymethyl)aminomethane) buffers, tromethamine, borate and any combinations thereof.

The amount of buffer substance added is, for example, that which is necessary to ensure and maintain a physiologically tolerable pH range, and in some embodiments maintain a pH which correlates to the pH of a human skin, which is typically higher than internal physiologic pH. The pH range is typically from 5 to 9, or from 6 to 8.2, or from 6.8 to 8.1.

Tonicity enhancing agents (structure-forming agents) are used to control the viscosity, flowability, pliability and spreadability (collectively presenting the consistency) of topical formulations, and hence may constitute a major excipient in the formulation, thus affecting gNO impregnation influencing the hydrophobic nature of the formulation.

Exemplary tonicity enhancing agents include, for example, ionic compounds, such as alkali metal or alkaline earth metal halides, such as, for example, CaCl₂, KBr, KCl, LiCl, NaBr, NaCl, or boric acid, non-ionic tonicity enhancing agents such as urea, glycerol, sorbitol, mannitol, propylene glycol, or dextrose.

Examples of preservatives include, but are not limited to, quaternary ammonium salts, such as cetrimide, benzalkonium chloride or benzoxonium chloride, alkyl-mercury salts of thiosalicylic acid, such as, for example, thimerosal, phenylmercuric nitrate, phenylmercuric acetate or phenylmercuric borate, parabens, such as, for example, methylparaben or propylparaben, benzoic acid, alcohols, such as, for example, chlorobutanol, benzyl alcohol or phenyl ethanol, guanidine derivatives, such as, for example, chlorohexidine or polyhexamethylene biguanide, or sorbic acid.

According to some embodiments of the invention presented herein, the gNO-sequestering topical formulation may further comprise other non-toxic excipients which may also improve the capacity of the formulation to sequester gNO.

Other non-toxic excipients include, for example, emulsifiers, wetting agents or fillers, such as, for example, the polyethylene glycols designated 200, 300, 400 and 600, 1000 or Carbowax designated 1000, 1500, 4000, 6000 and 10,000, complexing agents such as disodium-EDTA or EDTA, antioxidants such as ascorbic acid, acetylcysteine, cysteine, sodium hydrogen sulfite, butyl-hydroxyanisole, butyl-hydroxy-toluene or

α-tocopherol acetate; other stabilizers, such as a cyclodextrin, thiourea, thiosorbitol, sodium dioctyl sulfosuccinate or monothioglycerol; or yet other excipients, such as, for example, lauric acid sorbitol ester, triethanol amine oleate or palmitic acid ester.

Additional examples include, without limitation, surfactant systems effective to stabilize oil-in-water emulsions. These include, for example, non-ionic surfactants such as, but are not limited to, C-12 Laureth-23 BRIJ 35, C-16 Ceteth-10 BRIJ 56, C-16 Ceteth-20 BRIJ 58, C-16 IsoCeteth-20 Arlasolve 200, C-18 Steareth-20 BRIJ 78, C-18=Oleth-10 BRIJ 97, which can be obtained from ICI Surfactants, as well as surfactants such as, but not limited to, C-18 Steareth-10 Volpo S-10, C-18=Oleth-20 Volpo-20; C-18 Steareth-16 Solulan-16, C-18 Steareth-25 Solulan-25, which can be obtained from Amerchol Corp.; Alkyl polyglucose surfactants known as PLANTAREN, from Henkel; and Amphoteric surfactants such as, but are not limited to, alkyl amphodiacetates, alkyl amphodipropionates, betaines, sultaines, hydroxysultaines, and imidazolines, or salts thereof. It is recognized that other fatty acid condensates such as those formed with amino acids, proteins, and the like are also suitable.

Other components incorporated in the topical formulations include, but not limited to, silicic acid (e.g. the commercial product “Aerosil”), bentonites, modified montmorillonites, such as alkyl ammonium salts of montmorillonites (e.g., the commercial products “Bentone”), wherein the alkyl groups may contain 1 to 20 carbon atoms, (e.g., dimethyl dialkyl ammonium salts in which the alkyl groups contain 16 to 18 carbon atoms), cetostearylic alcohol and modified castor oil products (e.g., the commercial product “Antisettle CVP”).

The amount and type of excipient added is in accordance with the particular requirements of the application and indication, and is generally in the range from about 0.0001% to about 90% by weight of the total weight of the formulation.

According to some embodiments, the gNO-sequestering topical formulation presented herein comprises a therapeutically effective amount of gNO as described herein, a pharmaceutically, cosmeceutically or cosmetic acceptable carrier, and an additional therapeutically effective or pharmaceutical active agent which may be, for example, one or more of antimicrobial agents, antipruritic agents, anesthetic drugs, vitamins, anti-oxidants, antihistamines, antiallergic agents, anesthetic agents, antiphlogistic agents, non-steroidal anti-inflammatory agents, steroids, corticosteroids or any other drug.

The combined incorporation of gNO and an additional active agent (or active ingredient) in the topical formulation may result in additive therapeutic effect, a synergistic therapeutic effect and/or in widening the spectrum of activities imparted by the topical formulation.

Thus, for example, in cases where the additional active agent is an antimicrobial agent (as is gNO), the additional antimicrobial agent and the sequestered gNO can exhibit together an additive or even synergistic effect, thus resulting in a formulation with increased efficacy, or allowing to use lower amounts of the additional antimicrobial agent.

Alternatively, in cases where the additional agent is an antimicrobial agent which is, for example, an antibacterial agent that exhibits its activity on certain bacteria, or which is an antifungal agent, or any other antimicrobial agent other than an antibacterial agent, the combination of such an agent and the sequestered gNO, results in combination of pharmacological activities, and thus with a wider spectrum of activities.

Similarly, in cases where the additional agent is a cosmetic or cosmeceutic agent, the combination of such an agent with the antimicrobial activity of the sequestered gNO results in combined, widened, pharmacological activities.

Suitable antimicrobial agents, including antibacterial, antifungal, antiprotozoal and antiviral agents, for use in context of the present invention include, without limitation, beta-lactam drugs, quinolone drugs, bacitracin, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, farnesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole and mixtures thereof.

Suitable antipruritic agents include, without limitation, methdilazine and trimeprazine.

Non-limiting examples of anesthetic drugs that are suitable for use in context of the present invention include lidocaine, bupivacaine, chlorprocaine, dibucaine, etidocaine, mepivacaine, tetracaine, dyclonine, hexylcaine, procaine, cocaine, ketamine, pramoxine and phenol.

Non-limiting examples of anti-oxidants that are usable in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lysine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of vitamins usable in context of the present invention include vitamin A and its analogs and derivatives: retinol, retinal, retinol palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

Non-limiting examples of antihistamines usable in context of the present invention include chlorpheniramine, brompheniramine, dexchlorpheniramine, tripolidine, clemastine, diphenhydramine, promethazine, piperazines, piperidines, astemizole, loratadine and terfenadine.

For a non-limiting example, the pharmaceutically active agent can be a steroidal or non-steroidal anti-inflammatory agent for topical administration.

Representative examples of non-steroidal anti-inflammatory agents that are usable in this context of the present invention include, without limitation, oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamate, a flufenamic acid derivative, is particularly useful for topical application.

Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

For a non-limiting example, a gNO-sequestering formulation, according to some embodiments of the present invention, may contain diphenhydramine hydrochloride and zinc acetate as active ingredients in addition to nitric oxide, and a formulation containing the same can be used effectively as an anti-itch cream.

According to another embodiment, clotrimazole can be used as an active ingredient in addition to nitric oxide, which can be used effectively as a cream for treating vaginal yeast infection.

According to another embodiment, dimethicone, pramoxine hydrochloride and menthol can be used as active ingredients in addition to nitric oxide, and a formulation containing the same can be used effectively as an anti-itch cream.

According to another embodiment, hydrocortisone can be used as an active ingredient in addition to nitric oxide, and a formulation containing the same can be used as a steroid-based cream.

According to another embodiment, benzocaine and resorcinol can be used as active ingredients in addition to nitric oxide, and a formulation containing the same can be used effectively as an anti-itch cream.

According to another embodiment, carbomer 980, cetrimide, sorbitol, and a medium-chain triglyceride can be used as active ingredients in addition to nitric oxide, and a formulation containing the same can be used effectively as an ophtalmic gel for relief of eye dryness.

According to another embodiment, polymyxin and gramicidin can be used as active ingredients in addition to nitric oxide, and a formulation containing the same can be used effectively as an antibiotic cream.

According to another embodiment, coal tar can be used as an active ingredient in addition to nitric oxide, and a formulation containing the same can be used effectively as a gel for psoriasis relief.

According to another embodiment, moconazole can be used as an active ingredient in addition to nitric oxide, and a formulation containing the same can be used as an anti-itch cream.

As discussed herein, the gNO-sequestering formulations may be used in cosmetic and/or cosmeceutic applications and include cosmetically or cosmeceutically active agents in addition to nitric oxide, wherein the gNO can act as an active agent, as a cosmetic and/or cosmeceutic agent, as a preservative or as an antimicrobial agent.

In some embodiments, the gNO acts as a bacteriostatic, anti-contamination, stabilizing and/or sterilizaing agent, as described hereinabove, which prevents or reduces bacterial growth in the formulation.

In some embodiments, gNO acts as an antimicrobial agent, for preventing or reducing bacterial growth in or on the area treated by the cosmetic or cosmeceutic agent.

According to some embodiments of the present invention, cosmetic and/or cosmeceutic agents include, without limitation, anti-aging agents, anti-wrinkle agents, antioxidants, soothing and regenerating agents, vitamins, proteins, oily skin regulators, alpha and beta hydroxy acids, skin-lightening agents; emollients and humectants such as natural oils, natural butters and specialty emollients and humectants; emulsifiers such as oil-in-water and water-in-oil emulsifiers, instant cold emulsifiers and emulsion stabilizers; thickeners such as natural waxes, polymers and other thickeners and texturizers; botanical extracts; cleansing ingredients such as surfactants, conditioners and exfoliants; antiperspirants and deodorants; sun care and blocking ingredients such as sunscreens and self-tanners; fragrances and preservatives; and colors and pigments such as inorganic, organic and mica colors and pigments.

The gNO-sequestering formulation presented herein can be used effectively to treat a wide range of medical, cosmetic or cosmeceutic conditions, diseases and disorders by virtue of the therapeutic effect of gNO and/or by the therapeutic effect of the additional pharmaceutically active agent.

Hence, according to an aspect of some embodiments of the present invention there is provided a topical formulation as described herein, identified for use in the treatment of such a medical, cosmetic or cosmeceutic condition. The topical formulation can be packaged in a packaging material and identified in print, in or on the packaging material, for use in the treatment of the indicated medical, cosmeceutical and/or cosmetic condition.

The condition, in some embodiments, is a condition that is treatable by topical or transdermal application.

According to an aspect of some embodiments of the present invention there is provided a use of the formulation as described herein, in the manufacture of a medicament or product for the treatment of a medical, cosmeceutical or cosmetic condition.

According to an aspect of some embodiments of the present invention there is provided a method of treating a medical, cosmeceutical or cosmetic condition treatable by topical or transdermal administration, the method comprising topically applying the formulation as described herein to a skin or mucosal tissue of a subject afflicted by the condition.

Medical, cosmetic or cosmeceutical conditions that can benefit from release of gaseous NO, when applied topically, with or without an additional active ingredient, include, but are not limited to, infections caused by pathogenic microorganisms, as discussed in further detail hereinbelow, wounds, particularly when associated with an infection, inflammation and/or pain, inflammatory diseases or disorders such as tinea pedis (athlete's foot), acne, tinea versicolor (a benign scaly skin condition), and thrombophlebitis, primary Raynaud's phenomenon (PRP), limited cutaneous systemic sclerosis (LCSSc), diabetic ulcer wounds, skin infections, eczema, rash, immunologically mediated systemic diseases, allergic response mediated systemic diseases, viral blisters such as one caused by herpes, sexual dysfunction such as erectile dysfunction, intrapartum fetal distress caused by uterine hypertonus, estrogen-depleted osteoporosis, anal fissure, anal fissure pain, post Hirschprung surgery (for the treatment of obstructive symptoms), acute strangulated internal hemorrhoids, and pain, including postoperative pain, complex regional pain syndrome type 1, and pain and symptoms of chronic extensor tendinosis of the elbow (tennis elbow), as well as damage to skin cells caused by UV radiation.

As gNO has been proven effective against many pathogenic microorganisms, its use in conjunction with another antimicrobial agent may be beneficial as it may provide an additive and/or synergistic effect of gNO and the other antimicrobial agent.

It is noted herein that NO-sequestering formulations, as described herein, which can be used to treat the a cosmetic or cosmeceutical condition, as described hereinabove, are effective per-se due to the beneficial effect conferred by nitric oxide.

In addition, NO-sequestering formulations, as described herein, containing additional active ingredients or not, enjoy the added beneficial antimicrobial, preservative, sterilizing and/or stabilizing effect conferred by nitric oxide.

Furthermore, gNO-sequestering formulations, as described herein, containing additional pharmaceutically active agent, may exhibit an additive beneficial effect, or synergistic effect, conferred by the co-presence of nitric oxide together with the additional pharmaceutically active agent, thereby widening the spectrum of activity and efficacy of each active ingredient when used alone, as described hereinabove.

It is to be further noted that an additive beneficial effect of gNO can also be attributed to its known vasodilating effect. As a result of such an effect, application of a topical formulation to an infected skin or mucosal tissue may increase the amount of white blood cells reaching the treated tissue, thereby enhancing the therapeutic activity of the formulation and/or providing an additional therapeutic effect to the effect exhibited by the formulation, as described herein.

Hence, according to some embodiments of the present invention, the gNO-sequestering topical formulation presented herein further comprises an antimicrobial agent, as an additional pharmaceutically active agent.

According to some embodiments of the present invention, a gNO-sequestering topical formulation presented herein is identified for use as an antimicrobial formulation.

In particular, the gNO-sequestering formulation presented herein can be used effectively to treat an infection caused by a pathogenic microorganism.

Hence, according to an aspect of embodiments of the present invention, there is provided a use of the gNO-sequestering formulation presented herein in the treatment of a microbial infection.

Accordingly, there is provided a method of treating an infection caused by a pathogenic microorganism, which is effected by topically applying the

gNO-sequestering formulation presented herein to an infected topical bodily site of a subject in need thereof.

Microbial infections include any infection caused by a pathogenic microorganism, including, bacterial infection, fungal infection, protozoal infection, viral infection and the like, including molluscum contagiosum (a viral infection of the skin or occasionally of the mucous membranes), fungal nail infections, and cutaneous leishmaniasis.

Topical bodily sites include skin, mucosal tissue, eye, ear, nose, mouth, rectum and vagina.

Hence, according to some embodiments of the present invention, the phrase “topically applying” is meant to encompass, without limitation, dermal applications, ophthalmic application, vaginal application, rectal application and intranasal application.

According to some embodiments of the present invention, the gNO-sequestering formulation presented herein can be concocted into any cosmetic, cosmeceutical and/or pharmaceutical form normally employed for topical application, such as creams, lotions, ointments, suppositories, powder or oily bases, dressings, solutions, gels, mousses, pastes, soaps, pads, wipes, patches, swabs and pledgets.

In some embodiments, the gNO-sequestering formulation presented herein is in a form of a cream.

In all the aforementioned forms, the gNO may be sequestered in any one of the carriers and excipients comprising the formulation, as well as in any solid support, component of addendum which forms a part of the delivery system of the formulation which can sequester gNO, such as fiber and fabrics of pads and wipes, polymeric backing of the patches and sticks of swabs.

In the context of embodiments of the invention, gNO is releasably sequestered in the topical formulation by charging the topical formulation with gNO.

Hence, according to an aspect of embodiments of the present invention, there is provided a process of preparing gNO-sequestering formulation presented herein, which includes exposing a topical formulation or a formulation carrier(s) to a gaseous nitric oxide-containing environment.

The process according to embodiments of this aspect of the present invention starts by placing a topical formulation in a chamber. As the topical formulation may be in a semi-solid or other non-solid state, as in the case of creams and ointments, the topical formulation is housed in a receptacle which is open or generally accessible to the ambient environment or the gNO-containing environment.

The chamber can be any tank, canister, vat, barrel, cask, hogshead, drum, case, wrapper, sheath, bag, compartment, vessel, container or receptacle which can serve as encasement for the receptacle housing the topical formulation in terms of size (internal size) and capacity to contain gases, and particularly gNO-containing environment. By capacity to contain, it is meant that the chamber is sealed to an extent that allows charging the topical formulation, and mechanically fit to sustain both negative and positive pressure. Such requirements typically translate to mechanical integrity for maintaining impermeability to gases, rigidity and/or durability to maintain negative and positive pressure, and the ability to be fitted with inlets and outlets without losing containment of gases, while maintaining the integrity of the receptacle containing the topical formulation disposed therein.

The chamber forms a part of a charging device, which is designed to carry out the process presented herein and includes inlets and outlets (a single or a plurality of each); tubes for connecting the inlets and outlets to the chamber, to external sources, pumps and exhausts and therebetween; various inlet and/or outlet valves; various optional inlet and/or outlet gauges for monitoring inflow and outflow of gases; and various optional absorbers and scavengers of undesirable contaminants, as well as gauges and indicators for monitoring the environment within the chamber at various steps of the process, as is further detailed hereinunder.

The process involves placing a topical formulation of interest (in a receptacle or without) into the chamber with the intention of loading the topical formulation with gNO.

The topical formulation disposed within the chamber can be any topical formulation, any carrier, excipient or additive of a topical formulation or a stock or raw material for preparing a topical formulation.

Once the topical formulation in disposed within the chamber, the process involves generating a reduced pressure in the chamber. The term “reduced pressure”,

is used synonymously with the terms “negative pressure” “under pressure” and/or “vacuum”. Evacuating the chamber from the ambient environment substantially evacuates (removes) gaseous substances and/or volatile substances found in the chamber as well as gaseous substances and/or volatile substances found in the topical formulation, at least to the extent of the most highly volatile constituents of the topical formulation which may be detrimental to the gNO charging process.

In the context of embodiments of the invention, the term “gaseous” refers to a state of a substance being a gas under certain conditions of pressure and temperature. For example, the melting point of nitric oxide at atmospheric pressure is −164° C. and the boiling point of nitric oxide is −152° C., hence nitric oxide is a gaseous substance at ambient conditions of pressure and temperature (i.e., room temperature). Oxygen, nitrogen and CO₂, present in an ambient atmosphere, are also gaseous substances. Humidity and moisture forming-water is essentially a mixture of vapor (gaseous water) and liquid water at ambient conditions of pressure and temperature, however under reduced pressure the equilibrium of gas-liquid of water would essentially shift towards the gaseous state. Hence, reducing the pressure in the chamber, according to some embodiments of the present invention, facilitates the removal of gNO degrading oxygen, moisture and humidity from the chamber prior to introducing gNO therein.

Generating a reduced pressure in the chamber can be effected by connecting any inlet or outlet of the charging device to an external source of reduced pressure, such as a vacuum pump, or a vacuum reservoir. A vacuum reservoir can be in the form of, for example, a container which has been evacuated from its content to posses a volume under reduced pressure which is substantially larger than the volume of the chamber, thereby being capable of taking-in (sucking in) at least a part of the ambient atmosphere of the chamber.

In the context of embodiments of the invention, the level of the reduced pressure which is reached in the chamber can be at any level of vacuum which is reasonably attainable in the charging device resented herein, and such that would not adversely alter a desired composition of the topical formulation substantially. According to some embodiments of the present invention, the depth of the vacuum can range from low vacuum levels (about 100 kPa to 3 kPa) to high vacuum levels (about 100 mPa to 100 nPa). In the context of embodiments of the invention, vacuum levels can be expressed as a negative value, namely by the value representing the difference in pressure relative to atmospheric pressure (which is 760 Torr, 101.23 kPa, 14.7 psi or 1 atmosphere). Hence, according to some embodiments of the present invention, the reduced/negative pressure attained in the chamber can range from about −50 psi to −0.5 psi, and typically −10 psi for typical topical formulations. The unit “psi” is used in the context of some embodiments of the present invention, to denote a pressure difference, typically as recorded by a gage or device, from a reference pressure, typically atmospheric pressure. “0 psi” therefore denotes atmospheric pressure (e.g., ambient pressure), whereby “−X psi” is used to express negative pressure (under pressure).

In some embodiments of this aspect of the present invention, generating the reduced pressure in the chamber is effected for a time period of at least 1 minute, or for a time period that ranges from 1 minute to 60 minutes, or from 1 minute to 30 minutes, or from 1 minute to 20 minutes, or from 1 minute to 15 minutes, e.g., for 1, 2, 3, 4, 5, 6, 7, 8, 9, 10, 11, 12, 13, 14, 15, 16, 17, 18, 19 or 20 minutes, or for 30, 40, 50 or 60 minutes. During this period, the topical formulation is exposed to reduced pressure and in essence undergoes a purging process during which gases and volatile substances are substantially removed therefrom. The generation of reduced pressure in the chamber can thus be regarded as a degassing process or as a gas-evacuation process.

It is noted herein that water and gNO tend to interact such that the capacity of a moist topical formulation to sequester gNO is reduced compared to a substantially non-aqueous topical formulation. It is further noted herein that gNO is a relatively hydrophobic substance and hence the presence of water (moisture or humidity) can adversely interfere with sequestering gNO. Hence, it is desirable to reduce the humidity in the topical formulation prior to exposing the topical formulation to gNO-containing environment.

Oxygen and gNO also interact to produce reactive species such as nitrogen dioxide (nitrate), according to the reaction 2NO+O₂→2NO₂, which is a toxic brownish gas. Water, oxygen and gNO react to produce nitrite according to the reaction 4NO+O₂+2H₂O→4HNO₂. Nitrates and nitrites are known to participate is various reactions in vivo, in which toxic reactive oxygen species are formed. These reactions and subsequent reactions involving products of these reactions are commonly referred to herein as gNO degradation, and the process presented herein attempt to minimize this gNO degradation, by minimizing the presence of reactive species other than gNO.

Nitrates, nitrites, and any other species that are formed directly or indirectly by a reaction of nitric oxide with oxygen and/or water or humidity are referred to herein as “reactive species other than nitric oxide” or simply as “reactive species” and are meant to include nitrogen and/or oxygen-containing reactive species.

In some embodiments, generating reduced pressure in the chamber is effected so as to reduce the humidity level in the topical formulation/chamber by at least 50% of its original (ambient) level. According to some embodiments of the present invention, generating reduced pressure is effected so as to effect a decrease the humidity level in the topical formulation by more than 50%, more than 60%, 70%, 80%, 90% and up to 100% reduction in humidity, which is essentially desiccating the topical formulation to a level of humidity of almost zero or essentially zero humidity. Preferably, the amount of humidity (relative humidity) in the ambient environment prior to introducing gNO into the chamber is reduced such that the relative humidity in the chamber prior and during the introduction of the gNO-containing environment can be from about 0% to about 25%, or any amount therebetween, for example, about 2%, 3%, 4%, 5%, 6%, 7%, 8%, 9%, 10%, 11%, 12%, 13%, 14%, 15%, 16%, 17%, 18%, 19%, 20%, 21%, 22%, 23% or 24%.

In the context of oxygen found in the topical formulation and/or the interior of the chamber, generating reduced pressure in the chamber is effected so as to effect reduction of oxygen level in the topical formulation/chamber by at least 50% of its original (ambient) level. According to some embodiments of the present invention, generating reduced pressure may decrease the oxygen level in the topical formulation by more than 50%, more than 60%, 70%, 80%, 90% and up to 100% reduction in oxygen level, which renders the topical formulation essentially devoid of oxygen.

According to some embodiments of the present invention, generating reduced pressure in the chamber is effected so as to effect reduction of both oxygen and humidity levels to below 50%, or below 40%, 30%, 20%, 10% and down to essentially 0% of the ambient levels of each of oxygen and humidity before the exposure to gNO-containing environment.

According to some embodiments of the present invention, gNO sequestering topical formulations as described herein are substantially devoid of humidity and/or oxygen, and in any event comprise no more than 20% by weight of humidity, oxygen or both, optionally less than 15%, less than 10%, less than 5%, less than 2%, less than 1%, less than 0.5%, less than 0.1%, less than 0.05% and less than 0.01% and even less than 0.001% by weight of humidity, oxygen or both.

In some embodiments, topical formulations as described herein comprise no more than 1 ppm per gram of nitrogen-containing or oxygen-containing reactive species, as defined herein, preferably no more than 1 ppb per gram formulation.

In some embodiments of the present invention, exposing the topical formulation to reduced pressure is an optional step, particularly in cases of topical formulation which are sensitive to reduced pressure. In other cases the exposure to reduced pressure is minimized to ensure minimal alteration to the formulation and yet effective reduction of oxygen and moisture levels at least in the ambient environment in the chamber.

In some embodiments, once the topical formulation in placed within the chamber, the chamber is sealed so as to allow the application of reduced pressure therein for any desired length of time. After sealing and generating a reduced pressure in the sealed chamber, the ambient environment in the chamber can be replaced with a gNO-containing environment as described hereinbelow.

Once most of the ambient environment, which may contain undesired levels of water and oxygen which may react with gNO, has been substantially removed by generating reduced pressure from within the chamber and the topical formulation, the chamber is filled with a replacement environment which comprises gaseous nitric oxide. In some embodiments, the gNO-containing environment is an ambient environment comprising nitric oxide. The phrase “ambient environment comprising nitric oxide” and the phrase “gaseous nitric-oxide containing environment” refer equally to pure gNO or to any mixture of gNO and a carrier gas. A carrier gas can be any inert or otherwise biologically and chemically compatible gas such as, but not limited to, helium, argon, nitrogen gas and any combination thereof.

It is noted herein that filling the chamber with a gNO-containing environment can be performed by allowing a gNO-containing environment to flow into the chamber, namely, by simply connecting the chamber, in which reduced pressure was generated,

to a source of the gNO-containing environment. This gaseous environment will flow into the chamber due to pressure differences. In some embodiments, filling the chamber with a gNO-containing environment can be performed by pushing the gNO-containing environment into the chamber at an elevated pressure, or allowing the

gNO-containing environment to be sucked into the chamber by force of the vacuum present therein.

Filling the chamber substantially charges the topical formulation with gNO. The gNO-containing environment which is introduced into the chamber contains a predetermined concentration of gNO which is capable of impregnating the formulation with the desired effective amount of gNO, as discussed herein. The pre-determined concentration of gNO in the provided gNO-containing environment may range from about 0.05% to about 10%, or any amount therebetween, for example about 0.05%, 0.1%, 0.5%, 1%, 2%, 3%, 4%, 5%, 6%, 7%, 8%, 9% or 10% of the total amount of the gNO-containing environment, with the remaining environment being the carrier gas. The amount of gNO can alternately be expressed in parts per million (ppm), wherein ppm and percent values readily interconvert; for example 100 ppm is 0.01%, 1,000 ppm is 0.1%, 10,000 ppm is 1%, and 100,000 ppm is 10%. According to some embodiments of the invention, the concentration of gNO in the gNO-containing environment ranges from 160 ppm to 50,000 ppm. The gNO concentration within the gNO-containing environment can be determined by methods well known and widely practiced to those skilled in the art.

The gNO-containing environment may be provided (maintained, supplied as a continuous flow or as a single disbursement) for a predetermined amount of time that ranges from about 1 minute to about 24 hours, namely from about 1 minute to about 24 hours, or any period therebetween, for example about 1, 2, 5, 10, 15, 20, 30, 40, 50 or 60 minutes, or about 1, 2, 4, 6, 8, 10, 12, 16, 18 or 24 hours or higher, and any period of time therebetween.

A continuous flow of gNO-containing environment can be effected at a flow rate that ranges from about 1 cubic centimeter or milliliter per minute (cc/min) to about 2,000 cc/min or any rate therebetween, for example 1, 5, 10, 20, 30, 40, 50, 60, 70, 80, 90, 100, 250, 500, 750, 1,000 or 2,000 cc/min, or any rate therebetween. According to some embodiments, the flow rate ranges from about 250 cc/minute to 1,700 cc/min

The ability to control the flow rate, concentration of gNO and the duration of exposure serve as three finely tuned means to adjust the amount of gNO sequestered in the topical formulation. For example, a higher concentration of gNO in the carrier gas will result in a topical formulation sequestering a higher amount of gNO.

Maintaining the topical formulation exposed to the gNO-containing environment for a longer time will result in a topical formulation sequestering a higher amount of gNO. Similarly, increasing the flow rate of the gNO-containing environment through the chamber corresponds to increasing the amount of gNO which the topical formulation is exposed to, resulting in a topical formulation sequestering a higher amount of gNO. In some cases the topical formulation cannot sustain high vacuum levels to long or any duration of time, and in these cases controlling the amount of sequestered gNO will be achieved by concentration and flow rate of the gNO-containing environment.

It is noted herein that the goal amount of sequestered gNO in any given topical formulation or a constituent thereof can be achieved in more than one way, using all possible variables presented herein (such as, for example, gNO concentration, duration of exposure, flow of gNO-containing environment, degassing by vacuum, desiccation and oxygen purging).

It is, however, noted herein that the mode by which the desired amount of sequestered gNO is achieved in the topical formulation depends on the nature and the composition of the topical formulation. Higher volumes of topical formulations and/or topical formulations made of ingredients having a low intrinsic capacity to sequester gNO and/or topical formulations that require particularly high level of sequestered gNO would typically require higher loading time and/or higher duration and/or extent of reduced pressure and/or higher flowing rate of gaseous NO.

Undershooting the optimal parameters of the aforementioned variables will result in a topical formulation with sub-effective amount of sequestered gNO, and overshooting may damage the topical formulation or some of its ingredients and/or the chamber of the charging device.

According to some embodiments of the present invention, the concentration of gNO in the gNO-containing environment ranges from 150 ppm to 60,000 ppm.

According to some embodiments of the present invention, the concentration of gNO in the gNO-containing environment ranges from 800 ppm to 20,000 ppm;

the period of time of exposure to the environment in the chamber ranges from 1 to 600 minutes or overnight (10-24 hours); and/or the flow rate at which the environment is introduced into the chamber ranges from 0.5 liter/minute to 5 liter/minute.

Once exposure to gNO-containing environment is completed, the process, according to some embodiments of the present invention, involves evacuating the residual gNO-containing environment from the chamber, in preparation to opening the chamber and retrieval of the treated topical formulation from the charging device. Such a step can be effected by purging the chamber by flowing a carrier gas or ambient atmosphere into the chamber, by pumping the gNO-containing environment from the chamber with a vacuum pump or by a combination of pumping and purging.

Depending on the ingredients of the topical formulation, the amount of gNO in the environment, the duration of exposure to the environment during dosing and on various other factors as described hereinabove, the resulting gNO-sequestering topical formulation or at least portions thereof, are dosed with a predetermined therapeutically effective amount of gNO, as discussed herein, or at least 0.01 μM of gaseous nitric oxide per 1 gram of formulation.

As discussed hereinabove, the topical formulation used in the methodology described in these embodiments of present invention can be placed in a receptacle during the charging process as long as the receptacle does not impair the process of gNO loading to an extent that is not mitigated by means within the process (namely by process-controllable factors that can increase gNO uptake). This definition therefore makes the distinction between gas-permeable and non-gas permeable (gas-impermeable) receptacle materials, since the latter impairs the gNO charging process when the receptacle is sealed and gNO cannot defuse therethrough. An unsealed gas-impermeable receptacle having a topical formulation housed therein does not impair gNO penetration to the topical formulation and hence is regarded as an open or unsealed receptacle and not as an effective gNO barrier.

According to some embodiments of the present invention, the process presented herein can be effected with a receptacle-packaged topical formulation as well as with a bare (unpackaged) topical formulation. The receptacle per se can be made from gas-permeable or from gas-impermeable materials.

The term “non-gas permeable” or the equivalent “gas-impermeable”, as used herein, refers to an attribute of a substance which is capable of preventing the passage of gas molecule therethrough by flux, conveyance, diffusion or transportation. In some embodiments of the invention, these terms refer to impermeability of gNO, however, this term is also used to indicate impermeability of the non-gas permeable to other gases such as water vapors and oxygen. Without being bound by a particular theory, it is assumed that since gNO has a lower diffusion cross-section then water vapors or oxygen, a gNO-impermeable substance forming the non-gas permeable container or package will also be impermeable to water vapors and oxygen, and that a substance that is impermeable to water vapors and oxygen may still be permeable to gNO.

Examples of materials suitable for making gas-impermeable packaging receptacle include, without limitation, glass, glassy ceramics, metals, metallic foils, metallic-plastic composite foils and the likes and a combination thereof as composites or as parts in a complete gas-impermeable package. The gas-impermeable material for packaging receptacle can also comprise more than one layer of a polymer, a metal, a resin or a plastic, and in some examples the packaging can comprise a plastic-backed metallic foil, such as used in many air-tight packaging receptacles. A gas-permeable material can be combined with a gas-impermeable material to form a composite gas-impermeable package receptacle.

The term “gas-permeable” refers to a chemical attribute of a material, which allows gas molecules to pass therethrough by flux, conveyance, diffusion or transportation. “Gas-permeability” also refers to a mechanical attribute of a receptacle made from any material in the sense that the material can be shaped, designed, manufactured and processed to be impermeable, permeable and anywhere between the extremes (semi-permeable). A material can be made porous or caulked, shaped thin or thick, processes glassy or rough and the likes, all of which are examples of mechanical propertied that affect gas-permeability. In the context of embodiments of the present invention, semi-permeability in the sense of rate of permeation is regarded as gas-permeable, and semi-permeability in the sense of selectivity towards one gas species to another is regarded as gas-permeable if it can permeate gNO, and gas-impermeable if it does not allow gNO to permeate therethrough.

Non-limiting examples of gas-permeable materials include low density polyethylene (LDPE), high density polyethylene (HDPE), polypropylene, medical grade paper, polycarbonate (PC), polyester, polyvinyl chloride (PVC), polyvinylidene chloride, perfluoroalkoxy (PFA), acrylobutylstyrene, polypropylene (PP) polytetrafluoroethylene (PTFE), polyacrylate, acrylic, polycarbonate, polyacrylonitrile-butadiene-styrene, polymethylpentene (PMP), polyacetal, polystyrene (PS) or the like.

In the context of embodiments of the present invention, a gas-impermeable package receptacle is also referred to interchangeably as a non-gas permeable enclosure or gas-impermeable enclosure.

Hence, according to some embodiments of the present invention, the topical formulation can be housed in a gas-permeable receptacle when disposed in the chamber during the process of generating a reduced pressure and charging the topical formulation with gNO; wherein the presence of the gas-permeable receptacle may or may not affect one or more parameters of the process. The end result of the process would be a gNO-charged topical formulation in a gNO-charged gas-permeable receptacle.

According to some embodiments of the present invention, the topical formulation is housed in an unclosed (unsealed, open) sealable gas-impermeable packaging receptacle (a non-gas permeable enclosure that can be closed and sealed) when disposed in the chamber during the process of charging it with gNO. According to such embodiments, the process is carried out essentially as described hereinabove, and at the end of the process the non-gas permeable enclosure is closed and sealed so as to constitute an intact and complete gNO barrier with respect to the gNO in the gNO-containing environment and the gNO-sequestering topical formulation enclosed therein, insulating the topical formulation from ambient environment, and allowing it to sustain prolonged storage under storage conditions.

By “storage conditions”, it is meant that the topical formulation is kept under conditions which maximize the length of time that maintain the amount of gNO essentially stable until it is removed from storage conditions. The term “storage conditions” encompasses any form of maintaining a gNO-containing environment in the immediate vicinity of the topical formulation, either in the charging device, the sealed chamber or in a non-gas permeable container or packaging receptacle.

Storage conditions also include maintaining an environment which is low in oxygen, moisture, heat or other factors which may reduce the levels of gNO sequestered in the topical formulation.

According to some embodiments of the present invention, the process presented herein can be carried out so as to manufacture a topical formulation impregnated with gNO and encased in a non-gas permeable receptacle package as follows:

placing a topical formulation housed in an open and sealable non-gas permeable receptacle within a chamber, wherein the receptacle may include any one or more of the optional humidity indicator, gNO indicator or desiccant;

optionally generating a reduced pressure in the chamber;

filling the chamber with a gNO-containing environment (exposure conditions); and then either

sealing the receptacle package under the exposure conditions; and

purging the chamber, opening the chamber and retrieving the sealed receptacle package encasing the topical formulation now sequestering gNO;

or

purging the chamber, opening the chamber and retrieving the open receptacle package encasing the topical formulation now sequestering gNO; and

sealing the receptacle package under ambient conditions.

Thus, according to an aspect of some embodiments of the present invention there is provided a packaged receptacle which comprises a material configured to house a topical formulation; an topical formulation disposed within the receptacle; and a gaseous nitric oxide-containing environment within the receptacle.

In some embodiments, the receptacle is a non-gas permeable receptacle package.

In some embodiments, the environment is an ambient environment, as described herein.

In some embodiments, the topical formulation in the package has gaseous nitric oxide sequestered therein.

The package as described herein can further include desiccants, nitric oxide indicators and other components, as described herein.

According to an aspect of some embodiments of the invention, there is provided charging device (or system), which comprises:

a chamber comprising an inlet for receiving a gaseous nitric-oxide containing environment and an outlet for releasing the gaseous nitric-oxide containing environment; and a topical formulation, or a receptacle containing a topical formulation, as described herein, disposed within the chamber.

Such a chamber can be utilized for practicing any of the processes described herein.

The chamber may be coated from within with a protective surface coating suitable for preventing gNO from reacting with the chamber's material. Such protective coating may include, as non-limiting examples, glass, chromium, stainless steel and other gNO-resistant substances and mixtures thereof.

The device may further comprise a nitric oxide indicator configured to undergo a color change suitable for visual assessment of whether the topical formulation or an environment surrounding it has been exposed to the nitric oxide.

In some embodiments, a gNO indicator comprises one or more gNO-sensitive substances that can produce a detectable signal when exposed to a certain or any level of gNO in an environment. For example, the detectable signal may be a color change of the gNO-sensitive substance which occurs upon exposure to gNO. Such a gNO indicator is suitable for a visual confirmation for sufficient exposure of the formulation to gNO. Exemplary gNO-sensitive substances include dyes such as, for example, 4-amino-5-methylamino-2′,7′-difluorofluorescein (DAF-FM). It is to be understood that other gNO-indicators are contemplated in the context of embodiments of the invention, such as other chemical indicators, electronic indicators, off-line indicators (a sample for measuring the exposure thereof to gNO at a site unrelated to the exposure site) and the likes.

In some embodiments, a desiccant can be placed in the chamber to absorb humidity. The desiccant can be disposed within the chamber and be configured to absorb humidity from the ambient environment.

Suitable desiccants include, but are not limited to, Drierite®, silica gel, calcium sulfate, calcium chloride, montmorillonite clay, molecular sieves, etc. The desiccant can reduce the amount of humidity in the ambient environment by about 75% to about 100%.

The charging device may also include at least one humidity indicator configured to indicate moisture saturation of the desiccant or humidity level in the chamber. A non-limiting example of a humidity indicator for indicating desiccant saturation include cobalt chloride (CoCl₂), which undergoes a color change from blue (anhydrous state) to purple (CoCl₂.2H₂O) to pink (Co(H₂O)₆]Cl₂) as the absorption of water increases.

Devices which are configured for practicing any of processes as described herein while generating reduced pressure in the chamber further comprise an outlet for generating a reduced pressure in the chamber.

In some embodiments, a package receptacle enclosing (or housing) the topical formulation is further included within the charging device. In some embodiments, the package is as non-gas permeable enclosure, utilized according to embodiments of the invention to provide a packaged gNO-sequestering topical formulation as described herein.

A process of preparing gNO-containing formulations, as described herein, can also be used for prolonging a self-life of a topical formulation or product, due to the sterilization effect and/or bacteriostatic effect and/or preservative effect, etc., imparted by the sequestered gNO, as described hereinabove.

In addition, a process as described herein can be used for sterilizing a topical formulation as described herein, as described hereinabove, and is useful, for example, in cases where other sterilization methods are incompatible with one or more of the ingredients in the formulation, as described hereinabove.

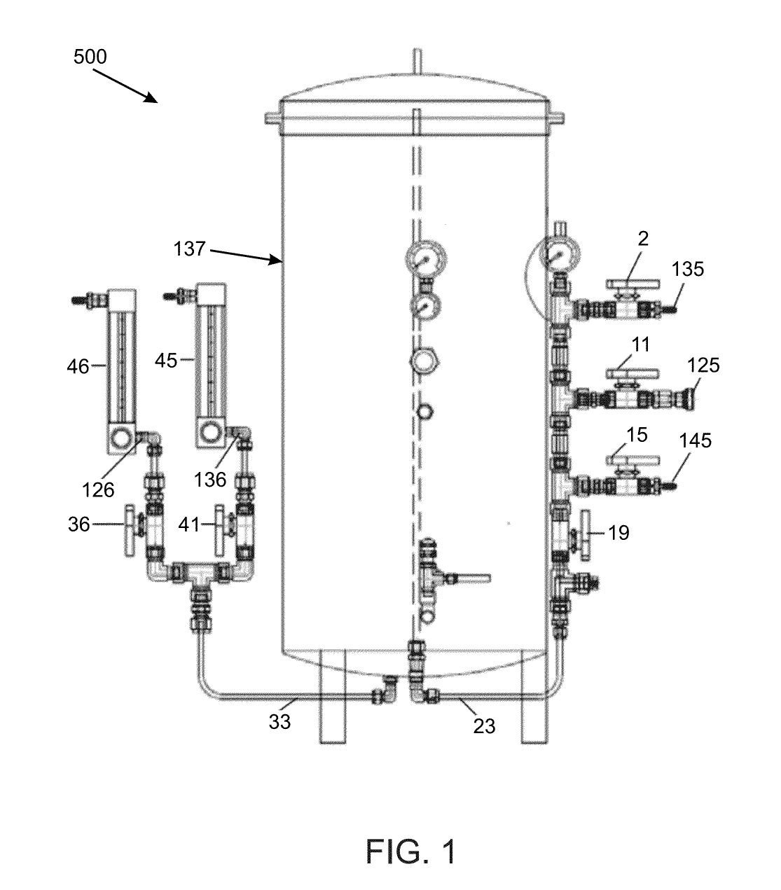

FIG. 1 presents a schematic illustration of an exemplary charging device 500 having a chamber 137, inlet 125 with valve 11, inlet 135 with valve 2, and inlet 145 with valve 15, inlets which may share a common tube 23 with valve 19 that connects to the chamber 137; outlet 126 with valve 36 and connect to purge flow rotomoter 46, outlet 136 with valve 41 and connect to purge flow rotomoter 45, outlets which may share a common tube 33 that connects to the chamber 137. Separate inlets 125, 135, 145 and outlets 126, 136 allow for gases (e.g. gNO, carrier gases) to enter and exit the chamber 137 at separate locations. Each of inlets 125, 135, 145 may also allow a vacuum creating mechanism (not shown) to have separate access to the chamber 137 for generating reduced pressure therein.

Such a setup makes it convenient for an operator of the charging device 137 to operate device 137 by removing the need to rehook vacuum, gNO and carrier gas sources to a single inlet at different stages of the process. Using the configuration presented in FIG. 1 allows each inlet 125, 135, 145 to be connected to a different source of gases or vacuum. For example, inlet 145 may serve as a vacuum port, inlet 125 may serve as an inlet for gNO or nitrogen gas, and inlet 135 may serve as a purging gas inlet for a carrier gas such as nitrogen gas, helium, argon, or any combination thereof.

The vacuum port 145 in FIG. 1 may be used to create a vacuum within chamber 137 so that the gNO introduced to chamber 137 does not react with any gases or moisture already contained within chamber 137 or within the topical formulation disposed therein (not shown). Chamber 137 may be pressured to any suitable pressure, for example, once the topical formulation has been disposed therein and chamber 137 has been sealed and evacuated substantially from the ambient environment therein, chamber 137 may be pressurized to 50 psig of gNO or of gNO mixed with a carrier gas such as helium, argon, nitrogen gas, and any combination thereof, helping to stabilize gNO.

Separate inlets 126, 136 in FIG. 1 may be connected by tubes to two rotometer kits 45, 46 which are flow meters that indicate flow rate and can be operated in parallel.

The gaseous nitric oxide (gNO) sequestering topical formulations presented herein can be prepared (charged with gNO) and be ready for use well in advance and kept in storage, or be prepared just prior to use, depending on the use, conditions and other preferences. In some cases, the formulation can be kept for extended lengths of time in a sealed chamber which is designed for charging its content with gNO, and then, prior to use, the device and formulation can be charged with gNO.

According to an aspect of embodiments of the invention, there is provided a charging device which includes a sealed chamber having a reduced pressure therewithin; and a topical formulation, as described herein (e.g., a medical device), disposed within the chamber.

Such a chamber can be utilized for charging the topical formulation with gNO, as described herein, and thus can further comprise an inlet configured for receiving a gaseous nitric oxide-containing environment and an outlet for releasing said gaseous nitric oxide-containing environment.

In some embodiments, such a chamber further comprises an outlet used for generating the negative pressure within the chamber.

Such a chamber can further comprise a non-gas permeable acceptable in which the topical formulation is positioned, as described herein. The device can further comprise desiccants and No-indicators as described herein.

It is expected that during the life of a patent maturing from this application many relevant nitric oxide sequestering articles will be developed and the scope of the phrase “nitric oxide sequestering topical formulation” is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and General Experimental Methods

The preparation of various nitric oxide-sequestering products, according to some embodiments of the present invention, is presented below. For clarity, it is noted that the terms “impregnated”, “charged”, “dosed” and “treated” are used interchangeably hereinbelow and throughout to denote nitric oxide-sequestering products.

Nitric Oxide Impregnation Method—A General Procedure

Topical formulations are exposed to 0.05-10% nitric oxide (Airgas Specialty Gases, Chicago, Ill.) in nitrogen (N₂) or argon (Ar) as a carrier gas for a period of 1-600 minutes or overnight (10-24 hours) in a sealed chamber, such as a stainless steel chamber. Topical formulations are placed in the chamber and the chamber is sealed. Prior to filling the chamber with nitric oxide, a reduced (negative) pressure of −10 psi (about −0.7 atmospheres) is maintained by a vacuum pump connected to the outlet of the chamber for a period of 1-15 minutes. When the negative pressure is reached, the pump is stopped and the outlet valve closed to maintain the negative pressure inside the chamber. Thereafter, the inlet valve connected to a nitric oxide-containing cylinder is opened to allow the nitric oxide in the carrier gas to flow and fill the chamber. The gas influx is stopped when ambient pressure is reached. After the desired exposure time, the gas inside the chamber is purged with air for at least 5 minutes through nitric oxide absorbing filters. Thereafter the chamber is opened and the exposed topical formulation is kept at room temperature.

NO-impregnation of Commercially Available Cream Products

The capacity to impregnate cream compositions was assessed with the commercially available pharmaceutical cream products presented in Table 1.

TABLE 1

| Brand | Manu- | | |
|---|---|---|---|
| Name | facturer | Active Ingredients | Use |
| Benadryl | McNeil | Diphenhydramine | Anti-itch |
| | | hydrochloride 1%, | cream |
| | | Zn acetate 0.1% | |
| Canesten | Bayer | Clotrimazole | Vaginal yeast |
| | | | infection |
| Gold Bond | Sanofi | Dimethicone 5%, | Anti-itch |
| | Aventis | Menthol 0.5%, | cream |
| | | Pramoxine | |
| | | hydrochloride 1% | |
| Hydrosone | Safeway | Hydrocortisone 0.5% | Steroid-based |
| | brand | | cream |
| Lanacane | Combe | 6% benzocaine, | Anti-itch |
| | | 2% resorcinol | cream |
| Liposic | Bausch and | Carbomer 980, Cetrimide, | Ophtalmic gel |
| | Lombe | Sorbitol, Medium-chain | for relief of |
| | | triglycerides | eye dryness |
| Polysporin | Johnson & | 10,000 U polymyxin B, | Antibiotic |
| | Johnson Inc | 0.25 mg gramicidin | cream |
| Psoriasin | Alba-Amco | Coal Tar 1.25% | Psoriasis gel |
| | | | relief |
| Vagisil | Combe | Moconazole | Anti-itch |
| | | | cream |

The cream products were exposed to 2% nitric oxide overnight (24 hours) using N₂ as a gas carrier in a sealed stainless steel chamber. Prior to the chamber filling with nitric oxide, a vacuum of −10 psi was obtained with a pump connected to the outlet of the chamber (about 5 minutes). When the negative pressure was reached, the pump was stopped and the outlet valve closed to maintain the negative pressure inside the chamber. Thereafter the inlet valve connected to a nitric oxide-containing cylinder (Praxair) was opened allowing the nitric oxide to flow and fill the chamber. The gas influx was stopped when pressure of 0 psi was reached. After the exposure time, the gas inside the chamber was released by purging it with air for at least 5 minutes through nitric oxide absorbing filters. Thereafter the chamber was opened and the exposed material was kept at room temperature.

NO-level Determination by Determination of Nitrites and Nitrates:

Nitric oxide has a half-life of a few seconds; therefore stable metabolites of nitric oxide (nitrates and nitrites) were measured using the Griess test [Green et al., Analysis of nitrate, nitrite and [¹⁵N] nitrate in biological fluids, Anal Biochem 126:131-138] to determine nitric oxide concentration in an aqueous medium.

Absorbance was translated to nitrite concentration using a standard curve prepared using samples with known nitrite concentrations. Nitrite production was converted into parts per million of nitric oxide as follows: nitric oxide ppm=(46×[NO₂])×0.65×10⁻³. Each μM (μmol/liter) was multiplied by the molecular mass of nitrite (46 grams/mol). This value was converted to ppm of nitric oxide, taking into account the difference in molecular weight (MW) between nitrites and nitric oxide (0.65), and the multiplication factor between grams and milligrams (10³).

Bacterial Culture Preparation:

The antibacterial activity of the charged products was assessed against microorganisms frequently associated with skin infections. The following microorganisms were tested:

Acinetobacter baumannii (ATCC BAA-747), Escherichia coli (ATCC 25922), Pseudomonas aeruginosa (ATCC 14210), Staphylococcus aureus (ATCC 25923), Methicillin-Resistant Staphylococcus aureus (MRSA) (ATCC 700698), and Streptococcus epidermidis (ATCC 14990).

Bacterial strains were cultured in Muller Hinton (MH) broth (B&D Biosciences) and maintained as stock on the same broth supplemented with 1.5 percents agar at 4° C. A microdilution assay was used to assess the antimicrobial activities using a 96-well plate and according to published protocols (NCCLS, 2006). Bacterial strains were cultured by shaking (200 rpm) at 37° C., overnight. The next day, the inoculum density was adjusted to be equivalent to 0.5 in the McFarland scale (McFarland, 1907) by using an optical density of 0.1 at 625 nm Serial dilutions up to 10⁻⁶ were performed using fresh MH broth.

### Example 1

### In Vitro Nitric Oxide Release from Commercial Creams

Weighted amount of each product (˜0.3 ml) was individually deposited at the bottom of sterilized 5 ml capped glass tubes by centrifugation (800 rpm, 3 minutes). Sterile double distilled water (2 ml) was aseptically added to each tube. Tubes were gently rocked in a rocker at 37° C. for 72 hours. Samples of 0.2 ml were taken every 24 hours, cleared by centrifugation at 15,000 rpm for 10 minutes, and the level of nitric oxide release was quantified as the total concentration of nitrites using the Griess test.

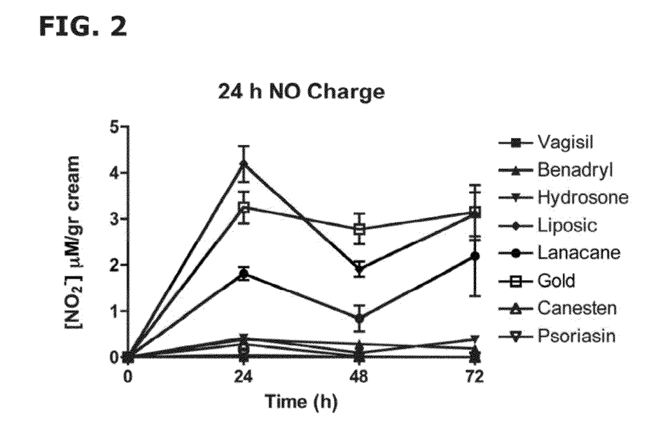

FIG. 2 presents the results obtained from two independent NO-release experiments conducted for commercial cream products which were exposed to 2% nitric oxide for 24 hours in a sealed stainless steel chamber, according to some embodiments of the present invention, wherein nitric oxide release was measured at time intervals of 24, 48 and 72 hours and the results are expressed in μM of nitrite per gram of product (mean values±standard deviation of two independent experiments).

As can be seen in FIG. 2, a time-dependent NO-release profile was observed with the products identified as Lanacane, Gold bond and Liposic, demonstrating that NO can be charged into the products and be released over time (NO can be releasably sequestered therein).

A similar experiment was conducted using the bacterial culture broth MH instead of water. 0.3 ml of each of Hydrosone, Polysporin, and Lanacane was individually deposited at the bottom of sterilized 5 ml capped glass vials by centrifugation (800 rpm, 3 minutes) and 0.5 ml of sterile MH was aseptically added to the vials. The capped vials were gently rocked in a rocker at 37° C. for 18 hours. Broth samples were extracted and cleared by centrifugation at 15,000 rpm for 30 minutes, and the level of nitric oxide release was quantified as the total concentration of nitrites using the Griess test. The results, obtained from two independent experiments, are presented in Table 2.

TABLE 2

| Product | NO₂⁻ (μM ± SD) |
|---|---|
| Hydrosone | 114.6 ± 11.9 |
| Polysporine | 39.9 ± 15.1 |
| Lanacane | 1728.9 ± 212.5 |

As can be seen in Table 2, nitric oxide was released from all the tested creams after treatment according to embodiments of the present invention, as measured by nitrite concentration in the supernatant, suggesting that nitric oxide can be impregnated within the components of the creams and released after contact with an aqueous solution.

### Example 2

### Stability of Commercial Creams Sequestering Nitric Oxide

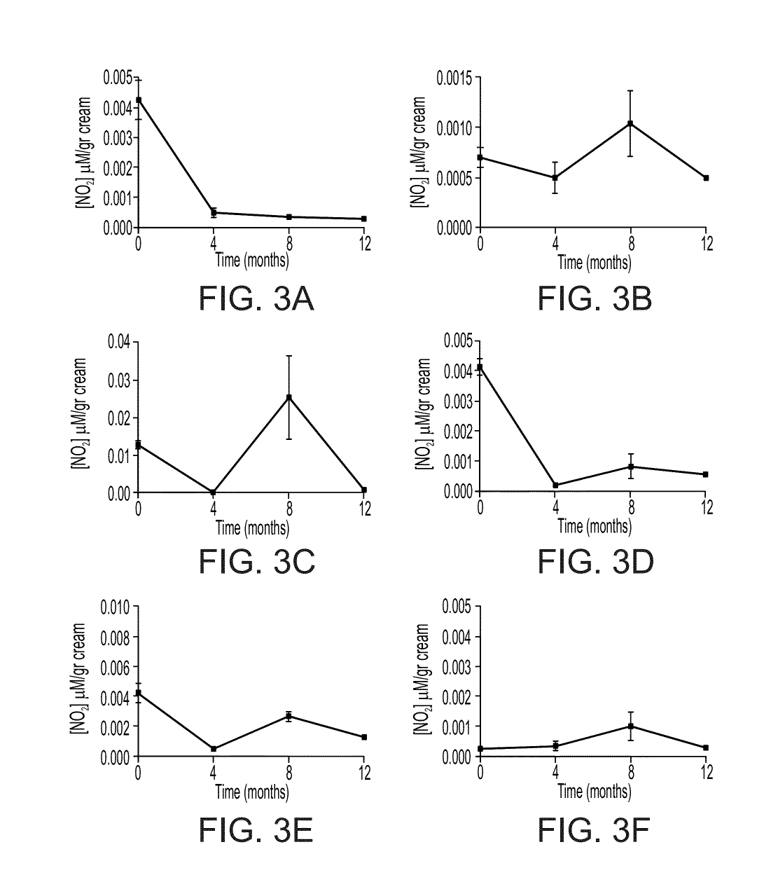

In order to study the capacity of an NO-impregnated cream to sequester NO while kept sealed in its original package after impregnation for a long period of time, impregnated cream samples were tested for NO content after a year from nitric oxide charging. Short term NO-release profiles from NO-impregnated commercially available pharmaceutical cream products, measured after one year from charging, are presented in FIGS. 3A-F.

FIGS. 3A-F present the results obtained from NO-release experiments conducted for commercial cream products which were exposed to 2% nitric oxide for 24 hours in a sealed stainless steel chamber, according to some embodiments of the present invention, wherein nitric oxide release was measured within one year of exposure to nitric oxide, at time intervals of 4, 8 and 12 hours, wherein FIG. 3A presents the NO release profile obtain for NO-impregnated Benadryl™, FIG. 3B for Canesten™, FIG. 3C for Hydrosone™, FIG. 3D for Gold Bond™, FIG. 3E for Lanacane™, FIG. 3F for Vagisil™ and the results are expressed in μM of nitrite per gram of product (mean values±standard deviation of three independent experiments).

As can be seen in FIGS. 3A-F, NO was released from the commercial cream and lotion products after the products were charged with NO and sealed for over a year.

### Example 3

### Antimicrobial Activity Assay of NO-Impregnated Creams

To facilitate the dispensing of the cream products, 2 ml glass made syringe tubes (Micro-Mate) previously sterilized by autoclaving, were filled with the exemplary commercially available cream products Hydrosone, Polysporin and Lanacane.

Each cream-filled tube and the respective plunger were wrapped individually with aluminum foil to maintain the sterility. Syringes were exposed to 2 percents nitric oxide for a period of 24 hours. Approximately 0.1 ml of each treated cream was settled down at the bottom of sterile 96-well plates by centrifugation of the plate at 800 rpm for 3 minutes. A final volume of 150 μl MH broth was added to each well. Non-treated creams were used as a control. Experiments were performed in triplicate.

Plates were sealed and gently rocked in a rocker at 37° C. for 18 hours. Thereafter, samples of 10 μl of each well were taken at time intervals of 0, 24, 48 and 72 hours, plated on MH agar, incubated at 37° C. overnight, and colony-forming units (CFU) were counted the next day.

Table 2 presents the results of the antimicrobial activity assay conducted with Hydrosone, an exemplary commercially available cream product, impregnated with NO according to embodiments of the present invention, expressed as CFU/ml±standard deviation, of AB denotes A. baumannii, EC denotes E. coli, PA denotes P. aeruginosa, MRSA denotes methicillin-resistant S. aureus, SE denotes S. epidermidis and ND denotes not determined

TABLE 3

| Time | AB | | EC | | PA | |
|---|---|---|---|---|---|---|
| (h) | − | + | − | + | − | − |
| 0 | 7.5 × 10⁶ ± | 7.5 × 10⁶ ± | 2.8 × 10⁷ ± | 2.8 × 10⁷ ± | 8.8 × 10⁷ ± | 8.8 × 10⁷ ± |
| | 1.5 × 10⁶ | 1.5 × 10⁶ | 2.5 × 10⁶ | 2.5 × 10⁶ | 7.5 × 10⁶ | 7.5 × 10⁶ |
| 24 | 8.8 × 10¹² ± | 0.0 | 3.7 × 10¹³ ± | 0.0 | 7.5 × 10¹³ ± | 7.5 × 10¹³ ± |
| | 4.2 × 10¹² | | 8.3 × 10¹² | | 2.9 × 10¹³ | 2.9 × 10¹³ |
| 48 | 5.7 × 10¹¹ ± | 0.0 | 3.5 × 10¹² ± | 0.0 | 3.3 × 10¹³ ± | 3.3 × 10¹³ ± |
| | 3.7 × 10¹¹ | | 5 × 10¹¹ | | 1.1 × 10¹³ | 1.1 × 10¹³ |
| 72 | ND | 0.0 | ND | 0.0 | ND | ND |
| Time | MRSA | | SA | | SE | |
| (h) | + | + | − | + | − | + |
| 0 | 8.8 × 10⁷ ± | 8.8 × 10⁷ ± | 8.1 × 10⁷ ± | 8.1 × 10⁷ ± | 3.5 × 10⁷ ± | 3.5 × 10⁷ ± |
| | 7.5 × 10⁶ | 7.5 × 10⁶ | 1.5 × 10⁶ | 1.5 × 10⁶ | 1.0 × 10⁶ | 1.0 × 10⁶ |
| 24 | 0.0 | 0.0 | 1.2 × 10¹⁴ ± | 0.0 | 6.2 × 10¹³ ± | 0.0 |
| | | | 5.9 × 10¹³ | | 5.4 × 10¹² | |
| 48 | 0.0 | 0.0 | 5.3 × 10¹² ± | 0.0 | 3.3 × 10¹² ± | 0.0 |
| | | | 1.7 × 10¹² | | 1.3 × 10¹² | |
| 72 | 0.0 | 0.0 | ND | 0.0 | ND | 0.0 |

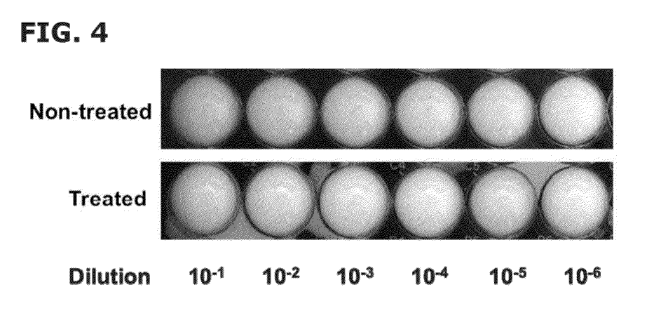

FIG. 4 presents a photograph of samples of Hydrosone treated with 2 percents nitric oxide for 24 hours, according to embodiments of the present invention, and un-treated samples of the same as controls, which were inoculated with a series of aliquots of Pseudomonas aeruginosa adjusted to an optical density of 0.1 at 625 nm by serial dilution with fresh MH growth broth, and allowed to develop cultures overnight, showing the appearance of a greenish color in the un-treated samples of Hydrosone indicating bacterial growth (presence of bacterial iron-binding metabolites), showing an original off-white color in the Hydrosone samples impregnated with nitric oxide, indicating no bacterial growth.

As can be seen in Table 3 and FIG. 4, bacterial growth of all tested strains after 24 hours exposure to the NO-impregnated commercial cream products was inhibited, suggesting that the released nitric oxide was able to kill all the microorganisms. Similar result was observed in the samples assessed after 48 and 72 hours.

Antibacterial activities of Lanacane and Polysporin were not determined as no bacterial growth was observed when strains were exposed to un-treated creams, presumably since Lanacane and Polysporin contain antibacterial agents.

### REFERENCES

Amadeu T P, S. A.-A.-C. (2008). J Surg Res., 149 (1), 84-93.
Arteaga-Troncoso, G., Villegas-Alvarado, A., & Belmont-Gomez, c. F.-H.-Z.-I. (2005). BJOG: an International Journal of Obstetrics and Gynaecology, 112, 1615-1619.
Briel J W, Z. D. (2000). Int J Colorectal Dis., 15 (4), 253-4.
Butler A R, G. C. (1987). Clin Chem., 33 (4), 490-2.
de Berrazueta J R, O. A. (1994). Rev Clin Esp., 194 (9), 734-5.
Doro, F G, Rodrigues-Filho, U., & Tfouni, E. (2007). Journal Of Colloid And Interface Science [J Colloid Interface Sci], 307 (2), 405-17.
El-Shimaa M. N. Abdel-Hafeza, G. E.-D.-R.-A. (2009). Bioorganic & Medicinal Chemistry Pages, 17 (11), 3829-3837.
Engel A F, E. Q. (2002). Br J Surg., 89 (1), 79-83.
Englander L, F. A. (2010). J Clin Aesthet Dermatol., 3 (6), 45-50.
Eroy-Reveles, A A, L., & Y, M. P. (2006). Journal Of The American Chemical Society [J Am Chem Soc], 128 (22), 7166-7.
Fenton C, W. K. (2006; 66). Drugs., 3, 343-9.
Finnen M J, H. A. (2007). Br J Dermatol, 157 (3), 494-500.
Friedman A, F. J.-2. (2009). Expert Opin Drug Deliv., 6 (10), 111.
Friedman, A., & Friedman, J. (2009). Expert Opinion On Drug Delivery [Expert Opin Drug Deliv]., 6 (10), 1113-22.
Ghaffari A, J. R. (2007). Wound Repair Regen., 15 (3), 368-77.
Ghaffari A, M. C. (2006). Nitric Oxide., 14 (1), 21-9.
Ghaffari A, N. D. (2005). Nitric Oxide., 12 (3), 129-40.
Gradoni L, A. P. (2004). Parassitologia., 46 (1-2), 101-3.
Gribbe O, G. L. (2008). Eur J Pharmacol., 578 (1), 51-6.
Groeneweg G, N. S. (2008). Clin J Pain., 24 (1), 89-92.
Gupta R, K. A. (2008). Biomed Mater, 3 (3), 034005.
Jowkarl, F., A, J., P, S., & M, &. R. (2010). Journal of Dermatological Treatment, 21 (2), 93-96.
Lauretti G R, O. A. (2000). Anesthesiology., 93 (4), 943-6.
Manookian C M, F. P. (1998). Am Surg., 64 (10), 962-4.
Meinhardt W, K. R. (1999). Drug Saf., 20 (2), 133-46.
Nablo, B. S. (2003). (BJ & MH) Journal Of Biomedical Materials Research. Part A [J Biomed Mater Res A], 67 (4), 1276-83.
Narin, G., Bulut, [symbol] ., Ülkü a, A., & Ülkü a, S. (2010). Applied Clay Science, 50 (4), 560-568.
Ormerod A D, W. M. (1999). Br J Dermatol., 141 (6), 1051-3.
Osman, I; MacKenzie, F; Norrie, J; Murray, H M; Greer, I A; Norman, J E (2006). American Journal Of Obstetrics And Gynecology [Am J Obstet Gynecol], 194 (4), 1012-21.
Paoloni J A, A. R. (2003). Am J Sports Med., 31 (6), 915-20.
Prado W A, S. V. (2002). Eur J Pharmacol, 19; 441 (1-2), 57-65.
Riccio D A, D. K. (2009). Biomaterials., 30 (27), 4494-502.
Sauaia M G, d. L. (2003). J Am Chem Soc., 125 (48), 14718-9.
Scatena R, B. P. (2010; 17). Curr Med Chem., 1, :61-73.
Schedin U, F. C. (1999). Br J Anaesth., 82 (2), 182-92.
Seabra A B, F. A. (2004). Br J Dermatol, 151 (5), 977-83.
Shishido, S. M. (2003). Biomaterials, 24 (20), 3543.
Silva de Santanaa, D., Pupoa, T., Sauaiab, M., & Santana da Silvaa R, a. L. (2010). International Journal of Pharmaceutics, 391 (1-2), 21-28.
Souto, S., P, P., C, R., AB, S., M, O., T, P., et al. (2010). Actas Urologicas Españolas [Actas Urol Esp], 34 (8), 708-12.
Suschek C V, K. V.-G.-B. (1999). J Biol. Chem., 5 (10), 130-7.
Tiryaki T, D. S. (2005). J Pediatr Gastroenterol Nutr., 40 (3), 390-2.
Weller R, F. M. (2006). Nitric Oxide., 15 (4), 395-9.
Weller R, O. A. (1998). J Am Acad Dermatol, 38 (4), 559-63.
Weller R, P. R. (2001). J Appl Microbiol, 90 (4), 648-52.
Weller R, S. A. (2003). Am J Physiol Cell Physiol., 284 (5), 1140-8.
Wimalawansa S J, G. J. (2009). J Clin Endocrinol Metab, 94 (9), 3356-64.
Wimalawansa, S. (2000). J Bone Miner Res., 15 (11), 2240-4.
Witte M B, K. T. (2002). Br J Surg, 89 (12), 1594-601.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-31. (canceled)

32. A topical formulation comprising a carrier having gaseous nitric oxide sequestered therein, said carrier being a base for a pharmaceutical, cosmetic or cosmeceutic product in a form selected from a cream, a lotion, an ointment, a wipe, a sponge, a solution, an emulsion, a paste, a gel, a patch, a pladget, a swab, a dressing or a pad.

33. The formulation of claim 32, wherein an amount of sequestered gaseous nitric oxide is at least 0.01 μM NO per gram of the formulation.

34. The formulation of claim 32, wherein said carrier is a pharmaceutically, cosmeceutic or cosmetic acceptable carrier.

35. The topical formulation of claim 34, wherein said carrier is selected from the group consisting of a hydrophobic carrier, a hydrophilic carrier and an amphiphilic carrier.

36. The topical formulation of claim 34, wherein said carrier is a hydrophobic carrier.

37. The topical formulation of claim 34, wherein said carrier is selected from the group consisting of a petroleum-based carrier, a mineral oil, a polyether, a polyvinyl, a cellulose derivative, an animal fat, a polymer of acrylic acid, a vegetable fat, a polysaccharide, a glycosaminoglycan, a saline solution, water and combinations thereof.

38. The formulation of claim 32, further comprising a pharmaceutically, cosmetically or cosmeceutically active agent.

39. The formulation of claim 38, wherein said active agent is an antimicrobial agent.

40. The formulation of claim 32, wherein said carrier comprises at least one additive or excipient.

41. The formulation of claim 40, wherein said additive or excipient is hydrophobic.

42. The formulation of claim 32, being in a form of a cream, a lotion, an ointment, a wipe, a sponge, a solution, an emulsion, a paste, a gel, a patch, a pladget, a swab, a dressing and a pad.

43. A method of treating a medical cosmetic or cosmeceutical condition treatable by topical or transdermal administration of a therapeutically, consmetically or cosmeceutically active agent, the method comprising topically applying the formulation of claim 32 to a skin or mucosal tissue of a subject afflicted by the medical, cosmetic or cosmeceutic condition.

44. The method of claim 43, wherein said medical condition is a microbial infection.

45. A process of preparing the formulation of claim 32, the process comprising exposing the topical formulation to a gaseous nitric oxide-containing environment.

46. The process of claim 45, wherein said exposing comprises:
placing the topical formulation in a chamber; and
filling the chamber with said nitric oxide-containing environment.

47. The process of claim 46, further comprising, prior to said filling, generating a reduced pressure in said chamber.

48. The process of claim 46, wherein said carrier is selected as being capable of sequestering therein at least 0.01 μM gaseous nitric oxide per gram.

49. The process of claim 46, wherein said carrier comprises at least one additive or excipient.

50. The process of claim 49, wherein said additive or excipient is hydrophobic.

51. The process of claim 45, wherein the topical formulation is in a form selected from the group consisting of a cream, a lotion, an ointment, a wipe, a sponge, a solution, an emulsion, a paste, a gel, a patch, a pladget, a swab, a dressing and pad.

52. The process of claim 45, wherein said carrier is a pharmaceutically, cosmeceutic or cosmetic acceptable carrier.

53. The process of claim 45, wherein said carrier is selected from the group consisting of a hydrophobic carrier, a hydrophilic carrier and an amphiphilic carrier.

54. The process of claim 45, wherein said carrier is a hydrophobic carrier.

55. The process of claim 45, wherein said carrier is selected from the group consisting of a petroleum-based carrier, a mineral oil, a polyether, a polyvinyl, a cellulose derivative, an animal fat, a polymer of acrylic acid, a vegetable fat, a polysaccharide, a glycosaminoglycan, a saline solution, water and combinations thereof.

56. A charging device comprising:
a chamber comprising an inlet for receiving a gaseous nitric-oxide containing environment and an outlet for releasing said gaseous nitric-oxide containing environment; and
a topical formulation disposed within the chamber, said topical formulation comprising a carrier, said carrier being a base for a pharmaceutical, cosmetic or cosmeceutic product in a form selected from a cream, a lotion, an ointment, a wipe, a sponge, a solution, an emulsion, a paste, a gel, a patch, a pladget, a swab, a dressing or a pad.

57. The charging device of claim 56, wherein said carrier is selected as being capable of sequestering therein at least 0.01 μM gaseous nitric oxide per gram.

58. The charging device of claim 56, wherein said carrier is selected from the group consisting of a hydrophobic carrier, a hydrophilic carrier and an amphiphilic carrier.

59. The charging device of claim 56, wherein said carrier is a hydrophobic carrier.

60. The charging device of claim 56, wherein said carrier is selected from the group consisting of a petroleum-based carrier, a mineral oil, a polyether, a polyvinyl, a cellulose derivative, an animal fat, a polymer of acrylic acid, a vegetable fat, a polysaccharide, a glycosaminoglycan, a saline solution, water and combinations thereof.

61. The charging device of claim 56, wherein said carrier comprises at least one additive or excipient.

62. The charging device of claim 61, wherein said additive or excipient is hydrophobic.

63. The charging device of claim 56, wherein the topical formulation is in a form selected from the group consisting of a cream, a lotion, an ointment, a wipe, a sponge, a solution, an emulsion, a paste, a gel, a patch, a pladget, a swab, a dressing and pad.

64. The device of claim 56, further comprising a package enclosing said topical formulation.

65. A charging device comprising:
a sealed chamber having a reduced pressure therewithin; and
a topical formulation disposed within the chamber, said topical formulation comprising a carrier, said carrier being a base for a pharmaceutical, cosmetic or cosmeceutic product in a form selected from a cream, a lotion, an ointment, a wipe, a sponge, a solution, an emulsion, a paste, a gel, a patch, a pladget, a swab, a dressing or a pad.

66. The charging device of claim 65, wherein said carrier is selected as being capable of sequestering therein at least 0.01 μM gaseous nitric oxide per gram.

67. The charging device of claim 65, wherein said carrier is selected from the group consisting of a hydrophobic carrier, a hydrophilic carrier and an amphiphilic carrier.

68. The charging device of claim 65, wherein said carrier is a hydrophobic carrier.

69. The charging device of claim 65, wherein said carrier is selected from the group consisting of a petroleum-based carrier, a mineral oil, a polyether, a polyvinyl, a cellulose derivative, an animal fat, a polymer of acrylic acid, a vegetable fat, a polysaccharide, a glycosaminoglycan, a saline solution, water and combinations thereof.

70. The charging device of claim 65, wherein said carrier comprises at least one additive or excipient.

71. The charging device of claim 70, wherein said additive or excipient is hydrophobic.

72. The charging device of claim 65, wherein the topical formulation is in a form selected from the group consisting of a cream, a lotion, an ointment, a wipe, a sponge, a solution, an emulsion, a paste, a gel, a patch, a pladget, a swab, a dressing and pad.

73. The device of claim 65, further comprising a package enclosing said topical formulation.
